(19) United States
(12) Patent Application Publication
(10) Pub. No.: US 2024/0034720 A1
(43) Pub. Date: Feb. 1, 2024

(54) Benzoxazine Derivatives Vitrimers

(71) Applicant: LUXEMBOURG INSTITUTE OF SCIENCE AND TECHNOLOGY (LIST), Esch-sur-Alzette (LU)

(72) Inventors: Pierre VERGE, Esch-sur-Alzette (LU); Antoine ADJAOUD, Esch-sur-Alzette (LU); Laura PUCHOT, Esch-sur-Alzette (LU); Acerina TREJO MACHIN, Esch-sur-Alzette (LU)

(21) Appl. No.: 18/256,785

(22) PCT Filed: Dec. 7, 2021
(86) PCT No.: PCT/EP2021/084608

§ 371 Date: Jun. 9, 2023

(30) Foreign Application Priority Data
Dec. 9, 2020 (LU) 102318

(51) Int. Cl. C07D 265/16; C08G 65/34; C08G 73/02

(52) U.S. Cl. CPC C07D 265/16; C08G 65/34; C08G 73/0233; C08G 2650/50; C08G 2650/42; C08G 2650/66; C08G 2650/64

(57) ABSTRACT

A process for producing a benzoxazine containing free aliphatic hydroxyl groups and monoester comprising the steps of: a) a reaction of a phenolic acid derivative with a monofunctional oligomer or molecule at a temperature of from 80° C. to 200° C., during 12 h-48 h, in a presence of a Bronsted type acid catalyst, resulting in a monophenol terminated oligomer or molecule and b) reaction of the monophenol terminated oligomer or molecule of step a) with a mixture of an amino-alcohol, a primary amine derivative and paraformaldehyde at a temperature range of from 80° C. to 100° C., from 1 h to 48 h, under stirring.

### CROSS-REFERENCE TO RELATED APPLICATIONS

The present invention is the US national stage under 35 U.S.C. § 371 of International Application No. PCT/EP2021/084608 which was filed on Dec. 7, 2021, and which claims the priority of application LU102318 filed on Dec. 9, 2020 the contents of which (text, drawings and claims) are incorporated here by reference in its entirety.

### FIELD

The invention is directed to the field of ester-containing benzoxazine derivatives vitrimers and to a process of manufacturing thereof and the use of the vitrimers in various applications.

### BACKGROUND

Composites are almost all the cases produced from thermoset resins, a material of choice for numerous applications because of their dimensional stability, mechanical properties and creep/chemical resistance. However, as a result of their permanent molecular architecture, they are impossible to recycle or to reprocess, and ends up in landfills.

A chemical way to tackle this drawback is offered by the introduction of exchangeable chemical bonds, leading to dynamic cross-links. Polymer networks containing such exchangeable bonds are also known as covalent adaptable networks (CANs) (W. Denissen et al.—Wim Denissen, Johan M. Winne and Filip E. Du Prez, Chem. Sci., 2016, 7, 30-38). CANs may be further classified into two groups depending on their exchange mechanism, either dissociative or associative. In the first, chemical bonds are first broken and then formed again at another place. Diels Alder reactions are the most common mechanism of dissociative CANs. In the second, polymer networks do not depolymerise upon heating, but are characterized by a fixed cross-link density. Covalent bonds are only broken when new ones are formed, making these networks permanent as well as dynamic. The first reported associative CANs (2005) were based on photo-mediated reactions by using allyl sulfides for instance. Later, a similar exchange mechanism was introduced by using alternative radical generators with trithiocarbonates.

In 2011, Leibler et al. (D. Montarnal, M. Capelot, F. Tournilhac and L. Leibler, Science, 2011, 334, 965-968) extended the field of associative CANs by adding a suitable transesterification catalyst to epoxy/acid or epoxy/anhydride polyester-based networks, resulting in permanent polyester/polyol networks that show a gradual viscosity decrease upon heating. Such a distinctive feature of vitreous silica had never been observed in organic polymer materials. Hence, the authors introduced the name vitrimers for those materials.

Vitrimers are portrayed as the third class of polymeric material owing to their outstanding features. The dynamic nature of the covalent network, arises from reversible chemical bonds, allows the material to be healed, recycled and reprocessed like thermoplastics. These exchange reactions are triggered by external stimulus, most frequently temperature. The viscosity of vitrimers gradually decreased upon heating providing malleability to the network while permitting internal stress to relax. Network integrity over the entire range of application ensures mechanical and solvent resistance.

Following the prototypal vitrimer developed by Leibler et al. in 2011 (previously mentioned), dynamic transesterification reactions demonstrated extensive interest over the last decade. These chemical exchanges induced at elevated temperatures between ester linkages and hydroxyl groups are responsible for topology rearrangements. Transesterification mechanism was implemented in cross-linked network to design self-healable, recyclable and reprocessable material with tunable properties.

Demongeot et al. (A. Demongeot, R. Groote, H. Goossens, T. Hoeks, F. Tournilhac and L. Leibler, Macromolecules, 2017, 50 (16), 6117-6127) adapted the vitrimer concept to commercially available thermoplastic. Cross-linked polybutylene terephthalate (PBT) vitrimer based on transesterification exchanges was successfully prepared by reactive extrusion. In addition to improving the manufacturing techniques and the potential scope of these networks, global environmental context urges the scientific community to promote sustainable polymer derived from naturally occurring feedstocks. Altuna et al. (F. I. Altuna, V. Pettarin and R. Williams, Green Chem., 2013, 15, 3360-3366) endeavoured to generate fully bio-based polyester showing properties reminiscent of vitrimers, starting from epoxidized soybean oil and an aqueous citric acid solution. Furthermore, Legrand et al. (A. Legrand and C. Soulid-Ziakovic, Macromolecules, 2016, 49, 5893-5902) enabled to extend the scalability of applications of vitrimer networks by developing a silica-reinforced epoxy vitrimer nanocomposites with enhanced properties.

Polybenzoxazines are a new type of thermoset with outstanding mechanical and thermal properties. As many other thermosets, they cannot be reshaped, re-processed nor recycled. A few examples have been reported showing a reasonable level of healability (L. Zhang, Z. Zhao, Z. Dai, L. Xu, F. Fu, T. Endo, X. Liu, ACS Macro. Lett. 2019, 8, 5, 506-511 and Arslan M., Kiskan B., Y. Yagci, Sci. Rep. 2017, 7, 5207). However, polybenzoxazine remains a class of high performance materials without any demonstration of vitrimers capabilities. Such sustainable vitrimer will widespread the use of polybenzoxazine towards smart coatings, reversible adhesives, or even recyclable matrix resins for composite materials.

### SUMMARY

The invention has for technical problem to provide a solution to at least one drawback of the above cited prior art.

The invention relates to an ester containing benzoxazine monomer of formula (I)

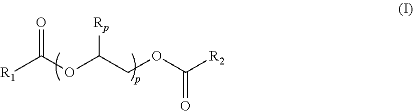

wherein
R₁ is

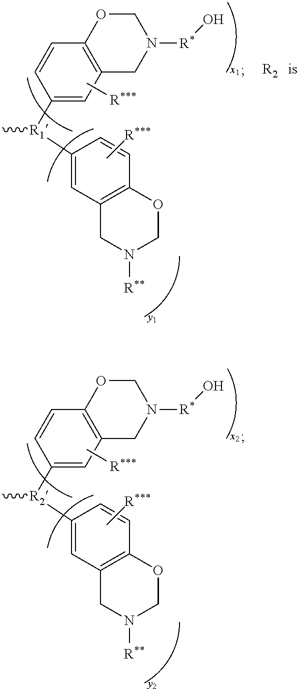

and
Rₚ is selected from the group consisting of H, a linear or branched C₁-C₆, preferably C₁-C₄, alkyl or alkoxy group, a linear or branched C₂-C₆, preferably C₂-C₄, alkenyl or alkylenoxy group, a substituted or unsubstituted linear or branched C₂-C₆, preferably C₂-C₄, alkynyl group, a linear or branched C₁-C₆, preferably C₁-C₄, alkyl or a C₂-C₆, preferably C₂-C₄, alkenyl substituted or unsubstituted phenyl group and

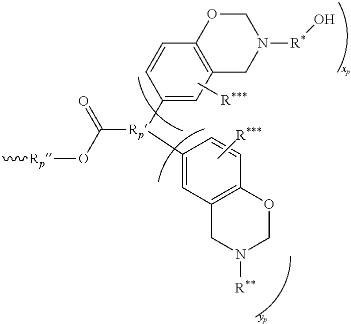

wherein
R₁ and R₂ of formula (I) are different;
x₁, x₂ and xₚ, independently, are of from 0 to 1; y₁=1-x₁; y₂=1-x₂ yₚ=1-xₚ, x₁, x₂ and xₚ values being not together 0;
p is 1-100;
R₁′, R₂′, and Rₚ′, independently, are selected from the group consisting of a —C-linear or branched C₁-C₆ alkyl or alkoxy group, a —C-linear or branched C₂-C₆ alkenyl or alkylenoxy group, a —C-substituted or unsubstituted linear or branched C₂-C₆ alkynyl group, and a —C-linear or branched C₁-C₆ alkyl or C₂-C₆ alkenyl substituted or unsubstituted phenyl group;
Rₚ″ is selected from the group consisting of a linear or branched C₁-C₆ alkyl or alkoxy group, a linear or branched C₂-C₆ alkenyl or alkylenoxy group, a substituted or unsubstituted linear or branched C₂-C₆ alkynyl group and a linear or branched C₁-C₆ alkyl or C₂-C₆ alkenyl substituted or unsubstituted phenyl group;
R* is selected from the group consisting of a linear or branched C₁-C₆ alkyl or alkoxy group, a cyclo(C₃-C₆alkyl) group, a heteocyclo(C₃-C₆alkyl) group, wherein the hetero atom is selected from N, S, and O, a linear or branched C₂-C₆ alkenyl or alkylenoxy group, a substituted or unsubstituted linear or branched C₂-C₆ alkynyl group, a linear or branched C₁-C₆ alkyl or C₂-C₆ alkenyl substituted or unsubstituted phenyl group, a (CH₂)ₙ₃-phenyl group and a —(CH₂)ₙ₃—O—(CH₂)ₙ₄ group, wherein n3 and n4, independently, are an integer from 1 to 10;
R** is the same as R* and further includes a member selected from the group consisting of a O—, N— or S—(CH₂)ₙ₃—CH—(CH₃)₂ group, a O—, N— or S—(CH₂)ₙ₃—(CHZ)ₙ₄—(C H₃)₂ group, a O—, N— or S—(CH₂)ₙ₃—(CHZ)ₙ₄—(CH₂)ₙ₃—CH₃ group, a O—, N— or S—(CHZ)ₙ₄—(CH₂)ₙ₃—CH₃ group, a O—, N— or S—(CHZ)ₙ₄—[(CH₂)ₙ₃—CH₃]₂ group, a O-substituted or unsubstituted C₂-C₆ linear or branched alkynyl group, a —(CH₂)ₙ₃—C≡N group and a polycyclic aromatic (PAH) or heteroaromatic hydrocarbon, such as naphthalene, anthracene, fluorene, phenanthrene, optionally substituted by a linear or branched C₁-C₆ alkyl or alkoxy group, a cyclo(C₃-C₆alkyl) group, a heterocyclo(C₃-C₆alkyl) group, a linear or branched C₂-C₆ alkenyl or alkylenoxy group, or by a substituted or unsubstituted linear or branched C₂-C₆ alkynyl group, wherein n3 and n4, independently, are an integer from 1 to 10, Z being selected from the group consisting of a linear or branched C₁-C₆ alkyl or alkoxy group, a linear or branched C₂-C₆ alkenyl or alkylenoxy group and a linear or branched C₁-C₆ alkyl or C₂-C₆ alkenyl substituted or unsubstituted phenyl group, and at least one O atom is present or not between two adjacent C,
R*** is selected from the group consisting of H, OH and a O-linear or branched C₁-C₆ alkyl group, and further includes a linear or branched C₁-C₁₅ alkyl group or a C₂-C₁₅ alkenyl group or

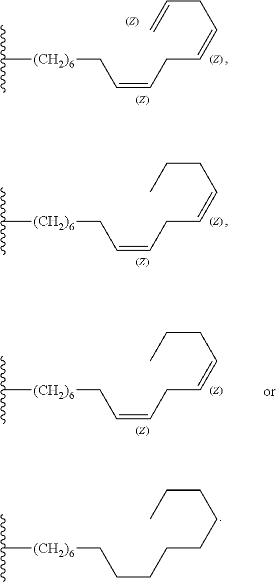

The ester-containing benzoxazine monomer of the invention is advantageously suited for obtaining polybenzoxazine derivatives vitrimers by a polymerization involving the benzoxazine ring opening and a self-polymerisation under heat, resulting to the polybenzoxazine derivatives vitrimers. Owing to the specific monomer starting product, the vitrimers of the invention exhibit self-healing, reshaping, reprocessability and recycling properties. For the rest of the document, benzoxazine vitrimers will always refer to the polymerized form of the ester-bond benzoxazine monomers.

The polybenzoxazine derivatives vitrimers properties are tightly connected to the properties of the ester-containing benzoxazine monomer.

As can be seen from formula (I), the monomer includes a benzoxazine ring moiety that allows the cross-linking of the monomer upon heating and that promotes the reprocessing of the obtained benzoxazine vitrimers thanks to the exchangeable ester bonds it forms once crosslinked. Benzoxazine gives thermosetting properties such as high-temperature and flammability performance, high strength, thermal stability, low water absorption, chemical resistance, low melt viscosities, and near-zero shrinkage.

The presence of a moiety consisting in ester bonds and free aliphatic hydroxyl groups are essential to form a dynamic and reversible network of the benzoxazine derivatives vitrimers, allowing the material to be recycled, reshaped and reprocessed. An amine terminated with a hydroxyl group allows to close the oxazine ring and allows the transesterification reactions. Accordingly, the essential features of the monomer of the invention rely on the benzoxazine-containing moiety, ester bonds and free aliphatic hydroxyl groups. The Tg of such polybenzoxazine can be of from 25° C. to 300° C. x₁, x₂ and xₚ, values, independently, can be of from 0.1 to 1 and y₁, y₂, and yₚ values are, respectively and independently, 1-x₁, 1-x₂ and 1-xₚ, more preferentially from 0.5 to 1. In some other embodiments, x₁, x₂ and xₚ values are not together 0, with x₁ and x₂ being not together 0.

Preferably, R* is selected from the group consisting of a linear or branched C₁-C₄ alkyl or alkoxy group, a linear or branched C₂-C₄ alkenyl or alkylenoxy group, an unsubstituted linear or branched C₂-C₄ alkynyl group, an unsubstituted phenyl group and a (CH₂)ₙ₃-phenyl group, a —(CH₂)ₙ₃—O—(CH₂)ₙ₄ group, wherein n3 and n4, independently, are an integer from 1 to 6; More preferably, R* can be selected from the group consisting of groups —CH₃, —(CH₂)ₙ₃—CH₃, —(CH₂)ₙ₃—CH—[(CH₂)ₙ₄—CH₃]₂, —C(CH₃)₃, (CH₂)ₙ₃—(C₆H₅), —(CH₂)ₙ₃—CH═CH₂, —(CH₂)ₙ₃—C≡CH, —(CH₂)ₙ₃—O—(CH₂)ₙ₄ wherein n3 and n4 independently are integer from 1 to 4, phenyl, and —(CH₂)₃-phenyl.

Preferably, R** is the same as R* and can further include a member selected from a O—, N— or S—(CH₂)ₙ₃—CH—(CH₃)₂ group, a O—, N— or S—(CH₂)ₙ₃—(CHZ)ₙ₄—(CH₃)₂ group, a O—, N— or S—(CH₂)ₙ₃—(CHZ)ₙ₄—(CH₂)ₙ₃—CH₃ group, a O—, N— or S—(CHZ)ₙ₄—(CH₂)ₙ₃—CH₃ group, a O—, N— or S—(CHZ)ₙ₄—[(CH₂)ₙ₃—CH₃]₂ group, a O-substituted or unsubstituted C₂-C₄ linear or branched alkynyl group and a polycyclic aromatic or a heteroaromatic hydrocarbon, wherein the hetero atom is selected from N, S, and O, such as naphthalene, anthracene, fluorene, furane, which can optionally be substituted by a linear or branched C₁-C₄ alkyl or alkoxy group, a linear or branched C₂-C₄ alkenyl or alkylenoxy group, a —(CH₂)ₙ₃—C≡N group, a cyclo(C₃-C₄alkyl) group, a heteocyclo(C₃-C₄ alkyl) group, or by a substituted or unsubstituted linear or branched C₂-C₄ alkynyl group, wherein n3 and n4, independently, are an integer from 1 to 6, Z being as above defined.

More preferably, R** can be the group R*, or can be selected from the group consisting of groups CH₃, —(CH₂)ₙ₃—CH₃, —(CH₂)ₙ₃—CH—[(CH₂)ₙ₄—CH₃]₂, —C(CH₃)₃, (CH₂)ₙ₃—(C₆H₅), —(CH₂)ₙ₃—CH═CH₂, —(CH₂)ₙ₃—C≡CH, O—(CH₂)ₙ₃—C≡CH, O—(CH₂)ₙ₃—C≡N, (CH₂)ₙ₃—C≡N, and —(CH₂)ₙ₃-substituted or unsubstituted furan, —(CH₂)-furfuryl, phenyl, and wherein n3 and n4, independently, are integer from 1 to 4.

R*** can be selected from the group consisting of H, OH and a O-linear or branched C₁-C₄ alkyl group, and can further include a linear or branched C₁-C₁₀ alkyl group or a C₂-C₁₀ alkenyl group or

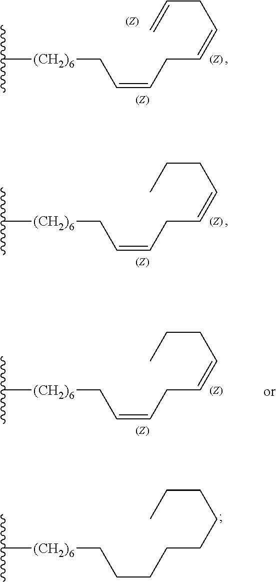

R*** can preferably be selected from the group consisting of H, OH and a O-linear or a branched C₁-C₃ alkyl group, and can further include a linear or branched C₁-C₆ alkyl group or C₂-C₆ alkenyl group or

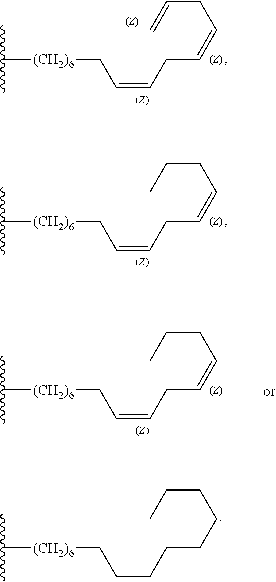

More preferably R*** is H.

The expression “substituted” as defined above, relates to the presence of some linear or branched alkyl groups in C₁-C₆.

The invention also relates to a process for synthesizing an ester-containing benzoxazine monomer of formula (I) comprising the following steps consisting of:
a) reacting a phenolic acid derivative of formula (II), comprising at least one R*** group on the phenolic ring:

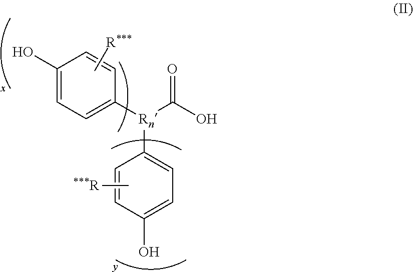

wherein x is of from 0 to 1, and y=1-x,
with a polyfunctional molecule or oligomer of formula (III)

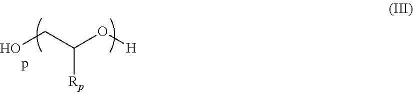

at a temperature of from 25° C. to 200° C., during 1 h-72 h, in the presence of a catalyst of Bronsted acid type, resulting in a phenol terminated oligomer or molecule (compound (IV)), and
b) reacting the compound (IV) with a mixture of:
   an amino-alcohol of formula (V):

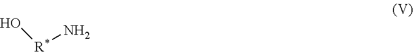

a primary amine derivative of formula (VI),

R**—NH₂ (VI), and
paraformaldehyde of formula (VII)

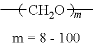

at a temperature range of from 80° C. to 100° C., from 1 h to 10 h, under stirring, for obtaining the compound of formula (I);
wherein R₁′, R₂′, Rₚ, R*, R**, R*** and p are, independently, as defined above, Rₙ′ being R₁′ or R₂′, R₁′ being different of R₂′, with the proviso that when at least one R*** of the phenolic acid derivative is in ortho position with regard to —OH group, then R*** is H.
x₁, x₂, xₚ and y₁, y₂, yₚ represent the proportion between benzoxazine groups when prepared from an aminoalcohol and the other amine(s). In other words, x₁, x₂, xₚ and y₁, y₂ and yₚ can be defined as

$${x}_{1} = \frac{{n}_{aminoalcohol ( R 1 )}}{{n}_{amines ( R 1 )}^{total}} \\ {x}_{2} = \frac{{n}_{aminoalcohol ( R 2 )}}{{n}_{amines ( R 2 )}^{total}} \\ {x}_{p} = \frac{{n}_{aminoalcohol ( Rp )}}{{n}_{amines ( Rp )}^{total}} \\ {y}_{1} = \frac{{n}_{amines ( R 1 )}}{{n}_{amines ( R 1 )}^{total}} \\ {y}_{2} = \frac{{n}_{amines ( R 2 )}}{{n}_{amines ( R 2 )}^{total}} \\ {y}_{p} = \frac{{n}_{amines ( Rp )}}{{n}_{amines ( Rp )}^{total}}$$

wherein n_{amine(R1)}^{total}=n_{amines(R1)}+n_{aminoalcohol(R1)}, and n_{aminoalcohol(R1)} being the number of aminoalcohol per R₁ group, n_{amines(R1)} represent the number of amines (excepting the number of aminoalcohol) per group R₁ and n_{amine(R1)}^{total}=n_{amines(R1)}+n_{aminoalcohol(R1)} is the total number of amino groups per group R₁;
wherein n_{amine(R2)}^{total}=n_{amines(R2)}+n_{aminoalcohol(R2)}, and n_{aminoalcohol(R2)} being the number of aminoalcohol per R₂ group, n_{amines(R2)} represents the number of amines (excepting the number of aminoalcohol) per group R₂ and n_{amine(R2)}^{total}=n_{amines(R2)}+n_{aminoalcohol(R2)} is the total number of amino groups per group R₂;
wherein n_{amine(Rp)}^{total}=n_{amines(Rp)}+n_{aminoalcohol(Rp)}, and n_{aminoalcohol(Rp)} being the number of aminoalcohol per Rₚ group, n_{amines(Rp)} represents the number of amines (excepting the number of aminoalcohol) per group Rₚ and n_{amine(Rp)}^{total}=n_{amines(Rp)}+n_{aminoalcohol(Rp)} is the total number of amino groups per group Rₚ.

The ester-containing benzoxazine monomer of the invention is advantageously suited for obtaining polybenzoxazine derivatives vitrimers by a polymerization involving the benzoxazine ring opening and a self-polymerisation under heat.

The Applicant has shown that the specific starting reactants are providing an ester-containing benzoxazine monomer, which in turn, after polymerization, is giving the polybenzoxazine derivatives vitrimers comprising polymerized benzoxazine.

The benzoxazine ring, obtained from the reaction of the specific compounds ((II)-(VII)) which allows the material to be cross-linked (processed) upon heating, helps the reprocessing thanks to the exchangeable and reversible ester bonds, and free aliphatic hydroxyl groups. Also, the benzoxazine ring moiety gives thermosetting properties such as high-temperature and flammability performance, high strength, thermal stability, low water absorption, chemical resistance, low melt viscosities, and near-zero shrinkage.

In the context of the invention, “derivative” in “phenolic acid derivative” means a compound bearing a phenolic acid moiety. Accordingly, “phenolic acid derivative” also means an organic compound bearing a phenolic acid group without being limitative.

The phenolic acid derivative (formula (II)) can be more preferably selected from the group consisting of mono-, di-, tri-hydroxybenzoic acid derivatives, anacardic acid derivatives, hydroxycinnamic acid derivatives, aliphatic X-hydroxyphenyl acid derivatives, wherein X is 2-4 and aliphatic diphenolic acid derivatives, or mixtures thereof.

Most preferred aliphatic mono-, di-, tri-hydroxybenzoic acid derivatives can be of formula (VIII)

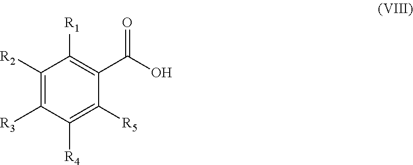

Wherein R′ₙ is omitted, and the R₁ to R₅ groups corresponding to R***, and one among R₁-R₅ is a hydroxyl group, then at least one H is in phenolic ortho-position, the rest being defined above.

Especially, in formula (VIII), at least one combination of R₁ to R₅ can be selected from the group consisting of:
R₁=OH, R₂=H, R₃=R₄=R₅=H or CH₃ or CH₂—CH₃ or CH₂—CH₂CH₃ or CH₂—CH(CH₃)₂,
R₂=OH, R₁=R₃=H, R₄=R₅=H or CH₃ or CH₂—CH₃ or CH₂—CH₂CH₃ or CH₂—CH(CH₃)₂,
R₃=OH, R₂=R₄=H, R₁=R₅=H or CH₃ or CH₂—CH₃ or CH₂—CH₂CH₃ or CH₂—CH(CH₃)₂,
R₄=OH, R₃=R₅=H, R₁=R₂=H or CH₃ or CH₂—CH₃ or CH₂—CH₂CH₃ or CH₂—CH(CH₃)₂,
R₅=OH, R₁=H, R₂=R₃=R₄=H or CH₃ or CH₂—CH₃ or CH₂—CH₂CH₃ or CH₂—CH(CH₃)₂.

Most preferred anacardic acid derivatives can be of formula (IX),

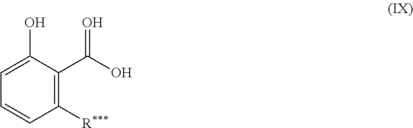

wherein R′ is omitted, and R*** is

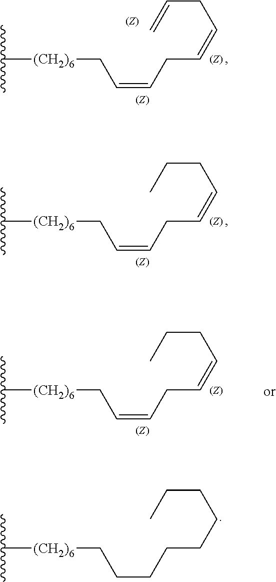

Most preferred hydroxycinnamic acid derivatives can be of formula (X)

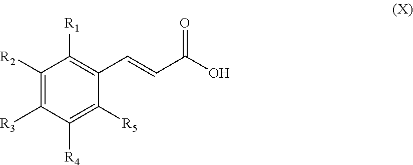

wherein R₁ to R₅ are corresponding to R***, and one among R₁-R₅ is a hydroxyl group and at least one H being in phenolic ortho-position, the rest being H and, optionally an aliphatic alkyl or alkoxy group of C₁-C₆.

Most preferred aliphatic X-hydroxyphenyl acid derivatives can be selected from the group consisting of aliphatic hydroxyphenyl acids (X=1), di-hydroxyphenyl acids (X=2), aliphatic tri-hydroxyphenyl acids (X=3) and aliphatic tetra-hydroxyphenyl acids (X=4), or mixtures thereof, of formula (XI)

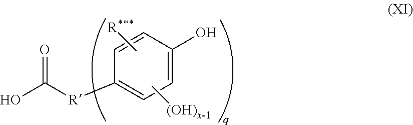

wherein R′ is selected from the group consisting of H, a —C-linear or branched C₁-C₆ alkyl or alkoxy group, a —C-linear or branched C₂-C₆ alkenyl or alkylenoxy group, a —C-substituted or unsubstituted linear or branched C₂-C₆ alkynyl group, and a —C-linear or branched C₁-C₆ alkyl or C₂-C₆ alkenyl substituted or unsubstituted phenyl group; and R*** is as defined previously.

The number of R*** in the ring is depending on the number of hydroxyl groups in the ring, and at least one R***, preferably of from 1 to 3, is H towards the phenolic ortho-position, and the integer q is comprised between 1 and 3.

Most preferred diphenolic acid derivatives are of formula (XII)

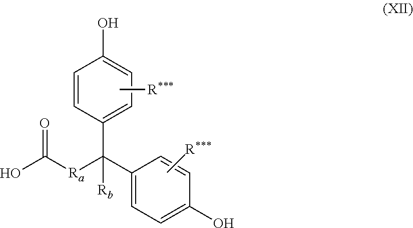

wherein
in the formula, —Rₐ—C—R_{b}— moiety is R′;
on each respective phenolic cycle, at least one R***, preferably of from 1 to 3, is H towards the phenolic ortho-position, and otherwise R*** is as defined previously, and R_{b} is selected from the group consisting of groups (CH₂)ₙ₅CH₃, (CH₂)ₙ₄-(aliphatic C₁-C₆ aliphatic alkyl or alkoxy substituted or unsubstituted phenyl group), wherein n5 is an integer from 1 to 12, preferably from 1 to 10, more preferably from 1 to 6, and (CH₂)ₙ₅(CH(CH₃)₂), and
Rₐ is selected from the group consisting of a (CH₂)ₙ₆ group, wherein n6 is an integer from 1 to 3, a CH(CH₂)ₙ₆(CH₃) group, a CH(CH(CH₃)₂) group and a C(CH₃)₂ group, the (CH₂)ₙ₆ group being the most preferred to lower the steric hindrance.

Most preferred is the 4,4-Bis(4-hydroxyphenyl)valeric acid (VA or DPA).

The polyfunctional molecule or oligomer compound of formula (III) is of importance for selecting the processing temperature of the benzoxazine polymer.

The compound of formula (III) can advantageously have 1-30, better 1-20, especially 1-10, p values, and can represent more preferably, when Rₚ=H, a polyethylene glycol (PEG) with a molecular weight (MW) in the range of from 4 MW of the C₂H₄O unit to 50 MW of the C₂H₄O unit, the MW of the C₂H₄O unit being classically of about 44.05 g/Mol. It is preferable to use commercially available PEG, for example PEG 200 to PEG 2200, as being easily available.

In the compound of formula (111), when Rₚ=H, p values can be of from 1 (ethylene glycol) to 3 (triethylene glycol—TEG).

In some other embodiments, the compound of formula (III) can be glycerol (Rₚ=CH₂OH).

The Bronsted acid type catalyst are those commonly used for a Fischer esterification include para-toluene sulfonic acid (p-TSA), anhydrous chlorhydric acid (HCl), phosphoric acid (H₃PO₄), methanoic acid (CH₃—CO₂H), sulfuric acid, tosylic acid, and Lewis acids such as scandium(III) triflate. The content of catalyst can typically be of from 0.5 wt % to 2 wt %.

The step a) can advantageously be carried out at a temperature in the range of 80° C. to 150° C., most preferably of from 100° C. to 140° C. for the best synthesis yields of higher than 95%, the chosen temperature being dependent on the nature of the reactants, i.e. the melting temperature of the reactant medium.

Advantageously, step a) is performed of from 12 h to 24 h for the highest yield of at least 95%, and the duration is based on the kinetic of the reaction.

The respective stoichiometry of starting reactants on step a), phenolic acid derivative:polyfunctional molecule or oligomer can preferably be 1.0-3.0 eq.:1.0 eq, resulting in an 1.0 eq. of phenol terminated oligomer or molecule.

The second step of the process, step b), corresponds to a Mannich condensation type reaction of the phenol terminated oligomer or molecule of step a) ((IV)) with the amino-alcohol (formula (V)), the primary amine derivative of formula (VI) and the paraformaldehyde (formula (VII)), optionally in presence of a catalyst. Thus, since step b) does not require the use of an external catalyst, step b) is implemented in an easier way.

Advantageously, the amino-alcohol of formula (V) includes R* group, a linear amino-alcohol with a primary amine moiety and an aliphatic hydroxyl moiety for obtaining with the highest yield and the best reaction conditions the oxazine ring.

The amino-alcohol of formula (V) can be more preferably selected from the group consisting of 2-aminoethanol, 2-amino-2-methylpropanol, 5-aminopentan-1-ol, heptaminol, 2-(2-Aminoethoxy)ethanol, and diglycolamine, or mixtures thereof.

The primary amine derivative includes the R** group as defined above.

In the context of the invention, “derivative” in “primary amine derivative” means a compound bearing a primary amine moiety. Accordingly, “primary amine derivative” also means an organic compound bearing a primary amine group without being limitative.

Primary amine derivatives are the same as R* and can be further selected from the group consisting in allylamine, methylamine, ethylamine, propylamine, butylamine, isopropylamine, hexylamine, cyclohexylamine, stearylamine, 2-aminofluorene, aminophenyl acetylene, propargyl ether aniline, 4-aminobenzonitrile, furfurylamine and aniline, or mixtures thereof.

The temperature range of step b) can preferably be of from 80° C. to 95° C., more allowing to obtain the highest conversion yields of at least 75%.

Advantageously, step b) is performed from 1 h to 8 h, preferably of from 1 h to 5 h, for the highest yield of at least 75%.

One advantage of the invention, is that step b) is performed without any catalyst.

The respective stoichiometry of starting reactants on step b), phenol terminated oligomer or molecule:amino-alcohol:primary amine derivative:paraformaldehyde can preferably be 1.0 eq.:x₁ (1.0 eq-18.0 eq):y₁ (1.0 eq-18.0 eq):2.0-36.0 eq; or 1.0 eq.:x₂ (1.0 eq-18.0 eq): y₂ (1.0 eq-18.0 eq):2.0-36.0 eq; or 1.0 eq.:xₚ (1.0 eq-18.0 eq): yₚ (1.0 eq-18.0 eq):2.0-36.0 eq resulting in an 1.0 eq. of the ester-containing benzoxazine monomer, wherein, independently, x₁, x₂ and xₚ=0-1, more preferably 0.1-1, or 0.5-1, and y₁=1-x₁, y₂=1-x₂ and yₚ=1-xₚ. It is also assumed that the higher are x₁, x₂ and xₚ, independently, the more efficient is the ROP.

The specific range stoichiometry is depending on the respective equivalent proportion of the amino-alcohol and of the primary amine derivative. It should be pointed out that there is a minimal quantity required for the reaction to occur. For instance, the relative molar % of amino-alcohol vs the relative molar % of primary amine derivative is 10 molar % vs 90 molar % respectively. It also means that primary amine can be omitted (0 molar %) and amino-alcohol can only be used instead (100 molar %). Besides, the selected stoichiometry ranges of both amino-alcohol/amine and paraformaldehyde preferably avoids the formation of either reaction linear and/or aliphatic by-products, such as oxazolidine, triaza derivatives, or condensation derivatives.

Preferentially, the whole process is performed with bio-based reactants.

The monoester-benzoxazine synthesis can most preferably be solventless, even though a solvent could be added for the dissolution of starting reactants. The process involves a one-step synthesis, which is one of the advantages of the invention.

Advantageously, the whole synthesis can generally not require any further monomer purification for the invention to be implemented. However, the purification of the monomer, if needed, can be performed by any known technic (vacuum, distillation etc.) The reaction mixtures of both steps a) and b) are stirred using a classical mechanical stirrer, or any non-limitative means.

The process can be implemented by any known means known to the one skilled in the art, using appropriate vessel either at lab scale or at industrial scale.

The invention also relates to a process for preparing a polybenzoxazine derivative vitrimer comprising the step of polymerization of an ester-containing benzoxazine monomer of the invention (formula (I)) or as obtainable by the above mentioned process at temperatures within the range of from 100° C. to 250° C. for 1 h to 24 h, for obtaining polybenzoxazine derivatives vitrimers.

In the context of the invention “derivative” means that the obtained vitrimer is obtained and derived through the polymerization of the benzoxazine monomer of the invention. Accordingly, “polybenzoxazine derivative vitrimer” or “polybenzoxazine vitrimer” have the same meaning.

According to the process for preparing the vitrimers of the invention, the polymerization step, which is a curing step, allows the benzoxazine ring to open and to react on itself to form a 3D network. Once cooled, the shape of the material is kept even after few months, typically 2-4 months. Once re-heated to at least 100° C. for a few minutes, the ester bonds are exchanging with the aliphatic hydroxyl group allowing the material to be reshaped, recycled, or reprocessed; while keeping structural integrity and number of covalent bound. Considering that Mannich condensation reaction is quantitative, nearly two hydroxyls groups could react with each ester bound through transesterification reaction (even after curing). The vitrimer behaviour strongly depend on the vitrimer glass transition (Tᵥ) also considered as the temperature where the transesterification reaction significantly increased. The vitrimer behaviours were demonstrated through several experiments. After the curing step, by heating the vitrimer above the Tᵥ, an initial shape of the vitrimer can be designed to other original shape. For example, vitrimers can be ground to a powder and can be reshaped or reprocessed at 150° C. in a couple of minutes. However its shape remains stable at room temperature.

The polymerization duration is depending on the curing temperature and/or on the nature of the ester-containing benzoxazine monomer. The polymerization temperature is selected for a given monomer to be higher than the temperature needed to synthesize the monomer. Generally, the higher the polymerization temperature, the shorter the curing duration. For example, when the temperature of the polymerization is 250° C., the curing duration can be of at least 1 h, and for a polymerization temperature of 100° C., the curing duration can be of no more than 24 h. Preferably, the curing temperature can be of from 140° C. to 200° C., more preferably of from 140° C. to 180° C., the latter range providing curing duration of from 1.5 h to 3 h, preferably of from 1.5 h to 2.5 h. The polymerization can be performed by any known heating means, such as laser beam and infrared beam.

The process can also include a post-polymerization step consisting of a heating step which can preferably be carried out at higher temperature than that the polymerization heating step.

The invention is also directed to a polybenzoxazine derivative vitrimer, that can be obtained by the above depicted process, exhibiting at least one of the following characteristics:
(i) Tᵥ values of from 100° C. to 250° C.; preferably of from 130° C. to 220° C., more preferably of from 130° C. to 190° C., and
(ii) Relaxation temperature values, Tᵥ values, of from 100° C. to 300° C., preferably of from 130° C. to 200° C., more preferably of from 130° C. to 180° C.

The vitrimers Tᵥ values are generally dependent from the nature and the content of the catalyst of step b), when present.

The relaxation temperatures typically correspond to the relaxation temperatures of the vitrimers after the appliance of a strain, for example a physical deformation such as a torsion, without the observation of vitrimers degradation.

Advantageously, the vitrimers can also exhibit at least one of the following characteristics selected from the group consisting of:
a relaxation time of from 0.5 s to 2 h, preferably of from 1 s to 1 h, more preferably of from 5 s to 50 min. The relaxation time is conventionally defined as the time for the sample to relax to a value corresponding 1/e (0,37) of its original modulus. Generally, the higher is the temperature, the shorter is the relaxation time. For example, the relaxation time is about 150 min-200 s at temperatures values of 120° C.-170° C., and of s 200, preferably 100 s-20 s, at temperature ranges of 150° C. to 200° C.

In some embodiments, the vitrimer can be deformed between 0.1% to 100% of its initial size;
an activation energy related to relaxation times can be of from 50 kJ/mol to 200 kJ/mol, preferably of from 70 kJ/mol to 170 kJ/mol, more preferably of from 100 kJ/mol to 160 kJ/mol; and
a processing temperature can be of from 100° C. to 250° C., preferably of from 130° C. to 250° C., more preferably of from 150° C. to 200° C., most preferably of from 150° C. to 170° C.

The vitrimers according to the invention can also very preferably exhibit the characteristics of behaving as a thermoset and/or an insolubility in many solvents, without been limited, such as water, CHCl₃, CH₂Cl₂, DMF, THF, aromatic solvents, such as toluene and/or xylene, ketones, alcohols or carboxylic acids. Swelling properties are observed as an extent of from 0 to 500% of the initial weight thereof. Swelling experiments can be carried out in various solvents, for example in acetone, chloroform and water to assess the formation of a cross-linked network. Among them, chloroform is the solvent in which the vitrimer shows the highest swelling ratio of about 100%. In acetone and water, the vitrimers swell of 40%-50% and 20%-30%, respectively.

The vitrimers of the invention present self-healing, reshaping, reprocessability, recycling and reversible adhesive properties.

The vitrimers can constitute an intermediate layer between at least two substrates, such as metal, polymer, glass and ceramic material. The resulting composite material can be prepared by setting at least one ester-containing benzoxazine monomer between the two considered substrates then curing at a temperature providing the vitrimer without altering the integrity of the substrates. Each substrate can be different from the other.

Metallic substrates are not limited, and can be of aluminium, iron, steel and the like.

Polymer substrates can be of polycarbonate, acrylic, polyamide, polyethylene or terephthalate.

Benzoxazine vitrimers can then be advantageously used in non-limited various fields of technologies, such electronics, aerospace, defense and automotive fields.

The invention also relates to a composition A comprising:
a) an ester-containing benzoxazine derivative of formula (I), and
b) at least one or more additional compounds of organic molecules types containing or not benzoxazine moieties.

Preferably, the organic molecules types can be polymers containing or not benzoxazine moieties.

The additional compound can be used to enhance the properties of either the monomer or the vitrimer (i.e. viscosity, mechanical and thermal properties), or both.

Polymers can be epoxy resins, bismaleimide resins, phenolic resins or benzoxazine resins, polyurethanes, polyamides, polyolefins, polyesters, rubbers. The ester-containing benzoxazine derivative of formula I can be used in a weight ratio from 0.1 to 80% of the final composition.

The compound of formula (I) can be used to provide vitrimer properties to the above mentioned polymers (self-healing, reprocessing, etc.).

The invention also relates to a composition B comprising:
a) an ester-containing benzoxazine monomer of formula (I), and
b) a material selected from the group consisting of fillers, fibers, pigments, dyes, and plasticizer.

The additional compound can be used to enhance the properties of either the monomer or the vitrimer (i.e. viscosity, mechanical and thermal properties), or both.

The additional compound could be carbon fibers, glass fibers, clays, carbon black, silica, carbon nanotubes, graphene, any known means for the thermal or the mechanical reinforcement of composites.

The invention also concerns a use of the vitrimer according to the invention as a reversible adhesive, sealant, coating or encapsulating systems for substrates selected from the group consisting of a metal, polymer, glass and ceramic material. Preferably, the metal and the polymer are as above defined.

The invention also relates to a use of the vitrimer according to the invention in 3D printing processes or in additive manufacturing processes.

### DRAWINGS

Other features and advantages of the present invention will be readily understood from the following detailed description and drawings among them.

FIG. 1 exemplarily shows a schematic synthesis reaction for obtaining ester-containing benzoxazine monomer of PEG-DPA/PA-mea/fa type, wherein R1′ and R2′ is —CH2-CH2- if either x1 or y1=0 and if either x2 or y2=0, and R1′ and R2′ is —CH2-C(CH3)- if either x1 and y1≠0 and x2 and y2≠0, and 0<x1≤0.75, 0<y1≤0.25 and 0<x2≤0.75, 0<y2≤0.25.

FIG. 2a) exemplarily displays the NMR spectrum of PEG-DPA/PA-mea/fa ester-containing benzoxazine monomer; FIG. 2b) exemplarily presents the DSC curve of PEG-DPA/PA-mea/fa.

FIG. 3 exemplarily shows the Stress relaxation curve of PEG-DPA-mea/fa vitrimer at 150° C.

FIG. 4 exemplarily shows a schematic synthesis reaction for obtaining the ester-containing benzoxazine monomer of PEG-DPA/PA-mea/a type, wherein R1′ and R2′ is —CH2-CH2- if either x1 or y1=0 and if either x2 or y2=0, and R1′ and R2′ is —CH2-C(CH3)- if either x1 and y1≠0 and x2 and y2≠0, and 0<x1≤0.75, 0<y1≤0.25 and 0<x2≤0.75, 0<y2≤0.25.

FIG. 5 exemplarily shows the Stress relaxation curve of PEG-DPA-mea/a vitrimer at 150° C.

FIG. 6 exemplarily shows a schematic synthesis reaction for ester-containing benzoxazine monomer named PEG-DPA/PA-aee/fa, wherein R1′ and R2′ is —CH2-CH2- if either x1 or y1=0 and if either x2 or y2=0, and R1′ and R2′ is —CH2-C(CH3)- if either x1 and y1≠0 and x2 and y2≠0, and 0<x1≤0.75, 0<y1≤0.25 and 0<x2≤0.75, 0<y2≤0.25.

FIG. 7 exemplarily represents the stress relaxation curve of PEG-DPA/PA-aee/fa vitrimer at 150° C.

FIG. 8 exemplarily shows a schematic synthesis reaction for obtaining the ester containing benzoxazine monomer of EG-DPA/PA-mea/ste type, wherein R1′ and R2′ is —CH2-CH2- if either x1 or y1=0 and if either x2 or y2=0, and R1′ and R2′ is —CH2-C(CH3)- if either x1 and y1≠0 and x2 and y2≠0, and 0<x1≤1.0, 0<y1≤1.0 and 0<x2≤1.0, 0<y2≤1.0.

FIG. 9 exemplarily shows the stress relaxation curve of poly(EG-DPA-mea/ste) vitrimer at 150° C.

FIG. 10 exemplarily shows a schematic synthesis reaction for obtaining the ester containing benzoxazine monomer of GLY-PHBA/PA-na/mipa/aee type, wherein R1′ and R2′ is Ø (phenyl) and 0<x1≤1.0, 0<y1≤1.0, 0<y1′≤1.0; 0<x2≤1.0, 0<y2≤1.0, 0<y2′≤1.0; and 0<xp≤1.0, 0<yp≤1.0, 0<yp′≤1.0.

FIG. 11 exemplarily shows the stress relaxation curve of poly(GLY-PHBA-na/mipa/aee) vitrimer at 150° C.

FIG. 12 exemplarily shows a schematic synthesis reaction for obtaining the ester containing benzoxazine monomer of PEG-DPA-mea/fa type, wherein R1′ and R2′ is CH2-CH2-C(CH3)- and 0<x1≤1.0, 0<y1≤1.0 and 0<x2≤1.0, 0<y2≤1.0.

FIGS. 13a), 13b) and 13c) exemplarily show the NMR spectrum of PEG-DPA-mea/fa ester-containing benzoxazine monomers.

FIG. 14a) exemplarily shows the DSC and Figure b) the isothermal rheology monitoring curves of PEG-DPA-mea/fa ester-containing benzoxazine monomers.

FIG. 15 shows the stress relaxation curves of poly(PEG-DPA-mea/fa) vitrimers at 150° C.

### DETAILED DESCRIPTION

All chemicals are commercially available and starting compounds, when applies, used as purchased.

### Example 1: Synthesis of an Ester-Containing Benzoxazine Monomer from 4,4-Bis(4-hydroxyphenyl)valeric acid (DPA) and 3-(4-Hydroxyphenyl)propanoic acid (PA) as Phenolic Acid Derivatives and Furfurylamine (fa) and Ethanolamine (mea) as Primary Amine with Aliphatic OH

The first step, step a), corresponds to a Fischer esterification between polyethylene glycol (PEG) (Mₙ=400 g·mol⁻¹, p=8-9, 1 eq, 2.8 g), 4,4-Bis(4-hydroxyphenyl)valeric acid (DPA) (0.85 eq, 1.73 g) and 3-(4-Hydroxyphenyl)propanoic acid (PA) (0.15 eq, 1.35 g) in presence of p-toluene sulfonic acid (pTSA) introduced in catalytic amount (1 wt %). PEG, DPA, PA and pTSA were reacted together in melt at 130° C. and agitated by mechanical stirring for 24 hours, to provide 4,4-Bis(4-hydroxyphenyl)valeric and 3-(4-Hydroxyphenyl)propanoic acid ester terminated polyethylene glycol (PEG-DPA/PA).

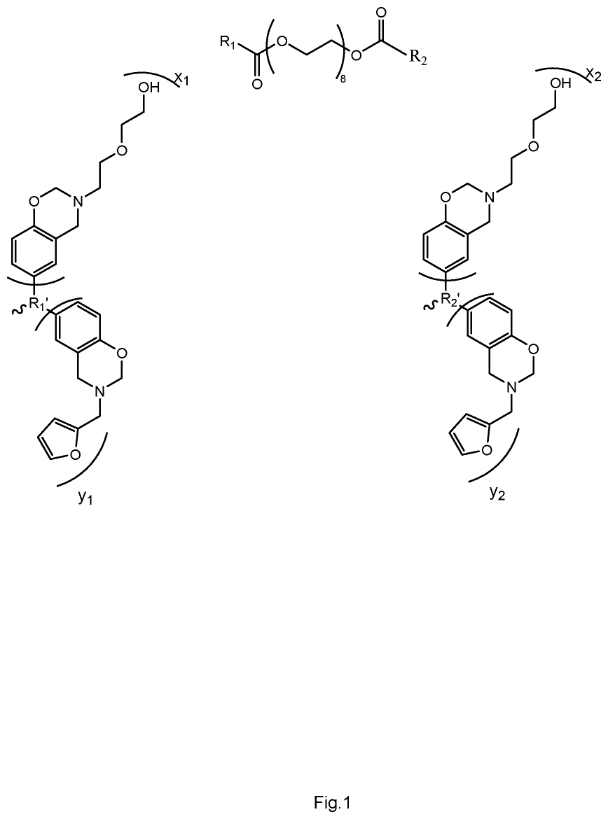

The second step, step b), corresponds to a Mannich condensation between 4,4-Bis(4-hydroxyphenyl)valeric and 3-(4-Hydroxyphenyl)propanoic acid ester terminated polyethylene glycol (PEG-DPA/PA) (1 eq, 5.4 g), furfurylamine (1.25 eq, 0.51 g) ethanolamine (mea) (1.75 eq, 0.97 g) and paraformaldehyde (PFA) (8.5 eq, 2 g). All these reactants were reacted together in melt at 85° C. and agitated by mechanical stirring for 8 hours to provide the ester-containing benzoxazine monomer named PEG-DPA/PA-mea/fa (see FIG. 1).

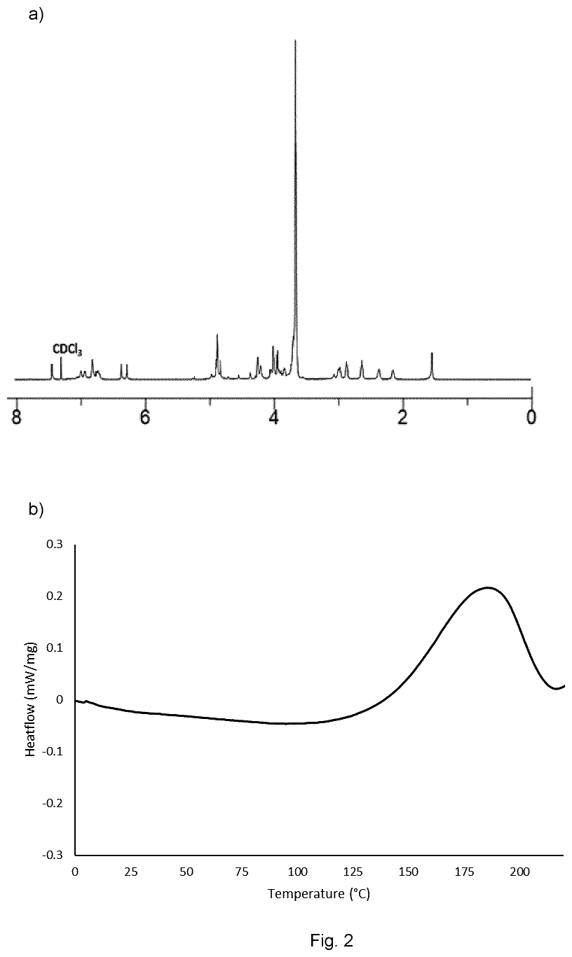

The FIG. 2a) displays the NMR spectrum (AVANCE III HD Bruker spectrometer) of PEG-DPA/PA-mea/fa ester-containing benzoxazine monomer in CDCl₃.

The DSC curve (Netzsch DSC 204 F1 Phoenix apparatus) shows an exothermic peak starting at a temperature of 125° C., with a maximum located at 180° C. (FIG. 2b)). This peak corresponds to the ring opening of the benzoxazine rings upon heating. The second peak corresponds to the thermal decomposition of the ester linkage confirmed by TGA experiment.

### Example 2: Vitrimer Synthesis from PEG-DPA/PA-mea/fa Benzoxazine Monomer

The PEG-DPA/PA-mea/fa benzoxazine monomer was cured 1 h at 170° C., allowing the benzoxazine rings to open and to react on themselves to form a 3D network vitrimer in a disk shape. The vitrimer behaviour of this sample was demonstrated through several rheology experiment.

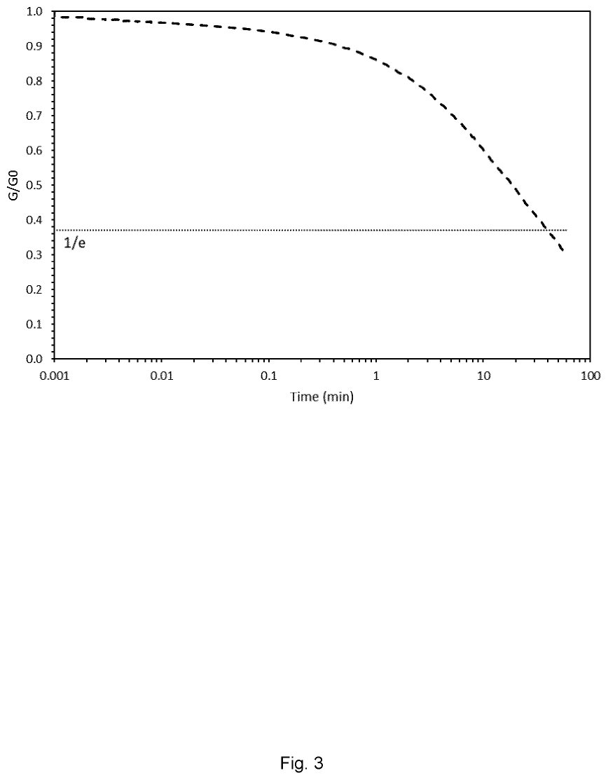

Viscoelastic properties of PEG-DPA-mea/fa vitrimer were studied by stress relaxation experiments recorded on Anton Paar Physica MCR 302 rheometer in plate-plate mode at 1% shear strain (FIG. 3). The relaxation time of the polymer was clearly noticeable and was recorded at 39.6 min at 150° C.

### Example 3: Synthesis of an Ester-Containing Benzoxazine Monomer from 4,4-Bis(4-hydroxyphenyl)valeric acid (DPA) and 3-(4-Hydroxyphenyl)propanoic acid (PA) as a Phenolic Acid Derivatives and Aniline (a) and Ethanolamine (mea) as Primary Amine with Aliphatic OH

Ester-containing benzoxazine monomer was synthesized in two stages.

The first step, step a), corresponds to a Fischer esterification between polyethylene glycol (PEG) (Mₙ=400 g·mol⁻¹, p=8-9, 1 eq, 2.8 g), 4,4-Bis(4-hydroxyphenyl)valeric acid (DPA) (0.85 eq, 1.73 g) and 3-(4-Hydroxyphenyl)propanoic acid (PA) (0.15 eq, 1.35 g) in presence of p-toluene sulfonic acid (pTSA) introduced in catalytic amount (1 wt %). PEG, DPA, PA and pTSA were reacted together in melt at 130° C. and agitated by mechanical stirring for 24 hours, to provide 4,4-Bis(4-hydroxyphenyl)valeric and 3-(4-Hydroxyphenyl)propanoic acid ester terminated polyethylene glycol (PEG-DPA/PA).

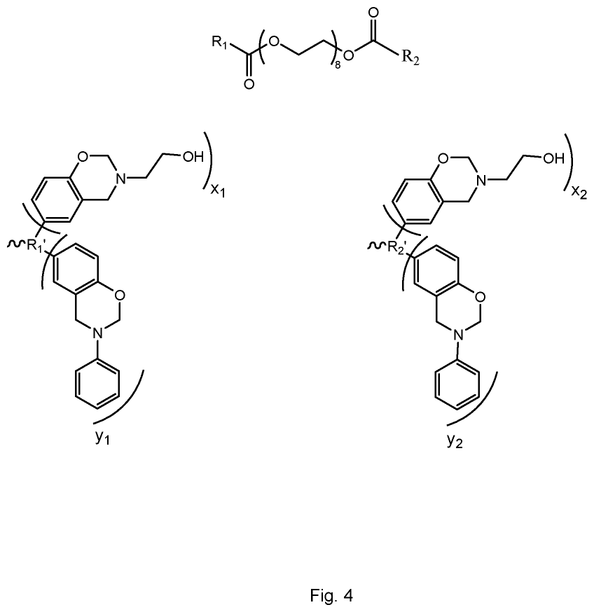

The second step, step b), corresponds to a Mannich condensation between 4,4-Bis(4-hydroxyphenyl)valeric and 3-(4-Hydroxyphenyl)propanoic acid ester terminated polyethylene glycol (PEG-DPA/PA) (1 eq, 5.4 g), aniline (1.25 eq, 0.79 g) ethanolamine (mea) (1.75 eq, 0.97 g) and paraformaldehyde (PFA) (8.5 eq, 2 g). All these reactants were reacted together in melt at 85° C. and agitated by mechanical stirring for 8 hours to provide the ester-containing benzoxazine monomer named PEG-DPA/PA-mea/a (see FIG. 4).

### Example 4: Vitrimer Synthesis from PEG-DPA/PA-mea/a Benzoxazine Monomer

The PEG-DPA/PA-mea/a benzoxazine monomer was cured 1 h at 170° C., allowing the benzoxazine rings to open and to react on themselves to form a 3D network vitrimer in a disk shape. The vitrimer behaviour of this sample was demonstrated through several rheology experiment.

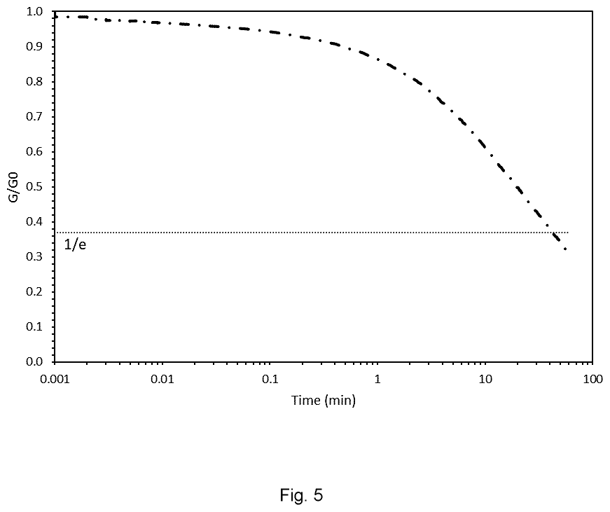

Viscoelastic properties of PEG-DPA/PA-mea/a vitrimer were studied by stress relaxation experiments recorded on Anton Paar Physica MCR 302 rheometer in plate-plate mode at 1% shear strain (FIG. 5). The relaxation time of the polymer was clearly noticeable and was recorded at 41.5 min at 150° C.

### Example 5: Synthesis of an Ester-Containing Benzoxazine Monomer from 4,4-Bis(4-hydroxyphenyl)valeric acid (DPA) and 3-(4-Hydroxyphenyl)propanoic acid (PA) as a Phenolic Acid Derivatives and Furfurylamine (a) and 2-(2-Aminoethoxy)ethanol (aee) as Primary Amine with Aliphatic OH

The first step, step a), corresponds to a Fischer esterification between polyethylene glycol (PEG) (Mₙ=400 g·mol⁻¹, p=8-9, 1 eq, 2.8 g), 4,4-Bis(4-hydroxyphenyl)valeric acid (DPA) (0.85 eq, 1.73 g) and 3-(4-Hydroxyphenyl)propanoic acid (PA) (0.15 eq, 1.35 g) in presence of p-toluene sulfonic acid (pTSA) introduced in catalytic amount (1 wt %). PEG, DPA, PA and pTSA were reacted together in melt at 130° C. and agitated by mechanical stirring for 24 hours, to provide 4,4-Bis(4-hydroxyphenyl)valeric and 3-(4-Hydroxyphenyl)propanoic acid ester terminated polyethylene glycol (PEG-DPA/PA).

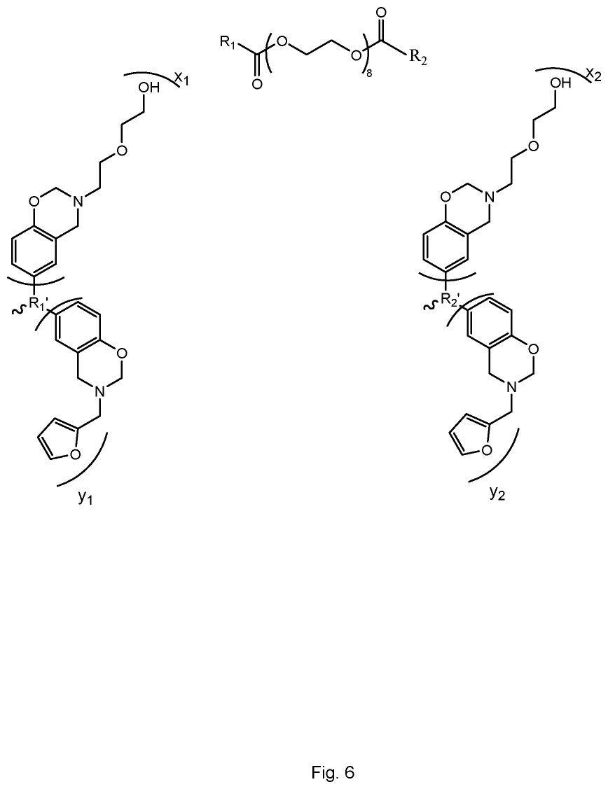

The second step, step b), corresponds to a Mannich condensation between 4,4-Bis(4-hydroxyphenyl)valeric and 3-(4-Hydroxyphenyl)propanoic acid ester terminated polyethylene glycol (PEG-DPA/PA) (1 eq, 5.4 g), furfurylamine (1.25 eq, 0.51 g), 2-(2-Aminoethoxy)ethanol (aee) (1.75 eq, 1.53 g) and paraformaldehyde (PFA) (8.5 eq, 2 g). All these reactants were reacted together in melt at 85° C. and agitated by mechanical stirring for 8 hours to provide the ester-containing benzoxazine monomer named PEG-DPA/PA-aee/fa (FIG. 6).

### Example 6: Vitrimer Synthesis from PEG-DPA/PA-aee/fa Benzoxazine Monomer

The PEG-DPA/PA-aee/fa benzoxazine monomer was cured 1 h at 170° C., allowing the benzoxazine rings to open and to react on themselves to form a 3D network vitrimer in a disk shape. The vitrimer behaviour of this sample was demonstrated through several rheology experiment.

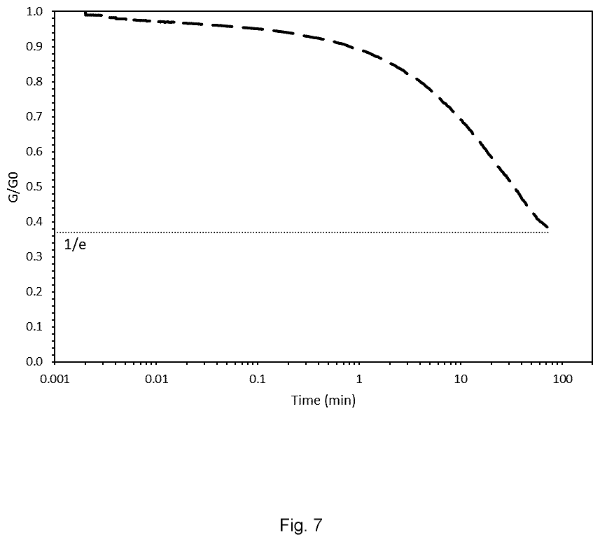

Viscoelastic properties of PEG-DPA/PA-aee/fa vitrimer were studied by stress relaxation experiments recorded on Anton Paar Physica MCR 302 rheometer in plate-plate mode at 1% shear strain (FIG. 7). The relaxation time of the polymer was clearly noticeable and was recorded at 75.6 min at 150° C.

### Example 7: Synthesis of an Ester-Containing Benzoxazine Monomer from Ethylene Glycol (EG), 4,4-Bis(4-hydroxyphenyl)valeric acid (DPA) and 3-(4-hydroxyphenyl)propanoic acid (PA) as Phenolic Acid Derivatives and Stearylamine (ste) and Mono-Ethanolamine (mea) as Primary Amine with Aliphatic OH

The first step, step a), corresponds to a Fischer esterification between ethylene glycol (EG) (1 eq, 5.00 g), 4,4-Bis(4-hydroxyphenyl)valeric acid (DPA) (1 eq, 23.07 g) and 3-(4-Hydroxyphenyl)propanoic acid (PA) (1 eq, 13.39 g) in presence of p-toluene sulfonic acid (pTSA) introduced in catalytic amount (1 wt %). EG, DPA, PA and pTSA were reacted together in melt at 130° C. and agitated by mechanical stirring for 24 hours, to provide 4,4-Bis(4-hydroxyphenyl)valeric and 3-(4-hydroxyphenyl) propanoic ester terminated ethylene glycol (EG-DPA/PA).

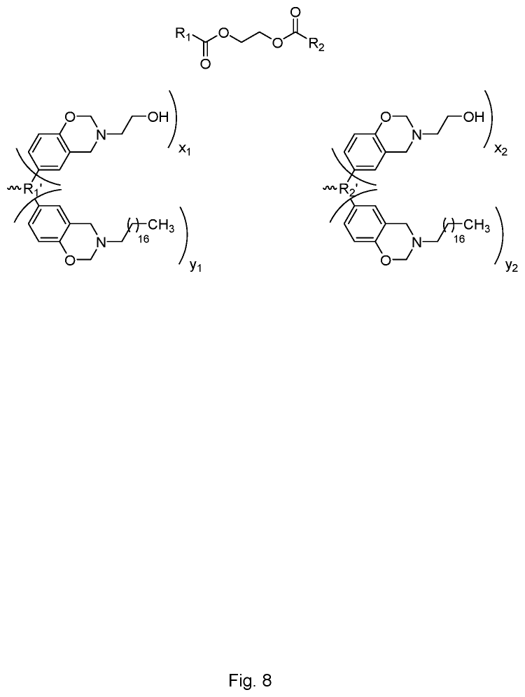

The second step, step b), corresponds to a Mannich condensation between 4,4-Bis(4-hydroxyphenyl)valeric and 3-(4-Hydroxyphenyl)propanoic ester terminated ethylene glycol (EG-DPA/PA) (1 eq, 5.00 g), stearylamine (ste) (1 eq, 2.82 g), mono-ethanolamine (mea) (1 eq, 0.64 g) and paraformaldehyde (PFA) (4 eq, 1.25 g). All these reactants were reacted together in melt at 85° C. and agitated by mechanical stirring for 8 hours to provide the ester-containing benzoxazine monomer named EG-DPA/PA-mea/ste (FIG. 8).

### Example 8: Vitrimer Synthesis from EG-DPA/PA-mea/ste Benzoxazine Monomer

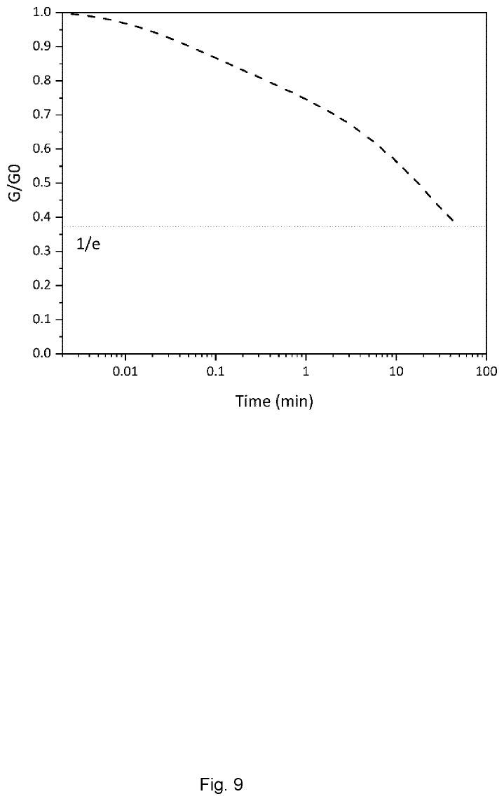

The EG-DPA/PA-mea/ste benzoxazine monomer was cured 1 h at 170° C., allowing the benzoxazine rings to open and to react on themselves to form a 3D network vitrimer in a disk shape. The vitrimer behaviour of this sample was demonstrated through several rheology experiment. Viscoelastic properties of poly(EG-DPA/PA-mea/ste) vitrimer were studied by stress relaxation experiments recorded on Anton Paar Physica MCR 302 rheometer in plate-plate mode at 1% shear strain (FIG. 9). The relaxation time of the polymer was clearly noticeable and was recorded at 49.4 min at 150° C.

### Example 9: Synthesis of an Ester-Containing Benzoxazine Monomer from Glycerol (GLY), 4-hydroxybenzoic acid (PHBA) as a Phenolic Acid Derivative and Nitroaniline (Na) and Mono-Isopropylamine (mipa) and 2-(2-Aminoethoxy)ethanol (aee) as Primary Amine with Aliphatic OH

The first step, step a), corresponds to a Fischer esterification between glycerol (GLY) (1 eq, 5.00 g), 4-hydroxybenzoic acid (PHBA) (3 eq, 22.50 g) in presence of p-toluene sulfonic acid (pTSA) introduced in catalytic amount (1 wt %). GLY, PHBA, and pTSA were reacted together in melt at 130° C. and agitated by mechanical stirring for 24 hours, to provide 4-hydroxybenzoic ester terminated glycerol (GLY-PHBA).

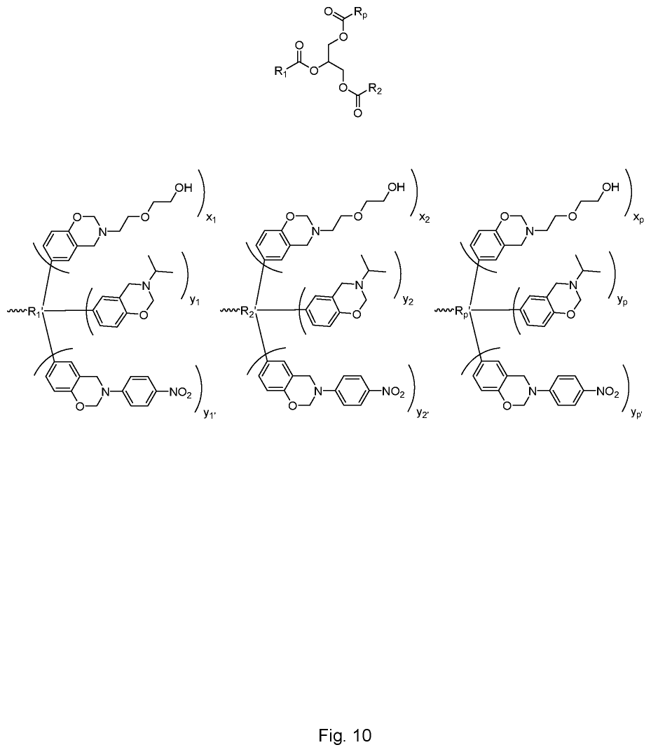

The second step, step b), corresponds to a Mannich condensation between 4-hydroxybenzoic ester terminated glycerol (GLY-PHBA) (1 eq, 5.00 g), nitroaniline (na) (1 eq, 1.53 g), mono-isopropylamine (mipa) (1 eq, 0.65 g), 2-(2-Aminoethoxy)ethanol (aee) (1 eq, 1.16 g), and paraformaldehyde (PFA) (6 eq, 1.99 g). All these reactants were reacted together in melt at 85° C. and agitated by mechanical stirring for 8 hours to provide the ester-containing benzoxazine monomer named GLY-PHBA-na/mipa/aee (FIG. 10).

### Example 10: Vitrimer Synthesis from GLY-PHBA-na/mipa/aee Benzoxazine Monomer

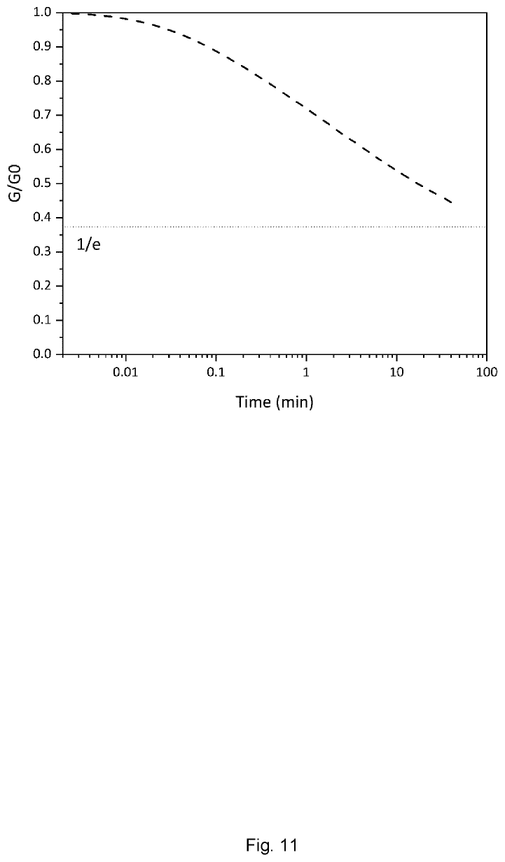

The GLY-PHBA-na/mipa/aee benzoxazine monomer was cured 1 h at 170° C., allowing the benzoxazine rings to open and to react on themselves to form a 3D network vitrimer in a disk shape. The vitrimer behaviour of this sample was demonstrated through several rheology experiment. Viscoelastic properties of poly(GLY-PHBA-na/mipa/aee) vitrimer were studied by stress relaxation experiments recorded on Anton Paar Physica MCR 302 rheometer in plate-plate mode at 1% shear strain (FIG. 11). The relaxation time of the polymer was clearly noticeable and was recorded at 88.1 min at 150° C.

### Example 11: Synthesis of an Ester-Containing Benzoxazine Monomer from Polyethylene Glycol (PEG), 4,4-Bis(4-hydroxyphenyl)valeric acid (DPA) as Phenolic Acid Derivative and Furfurylamine (fa) and Mono-Ethanolamine (mea) as Primary Amine with Aliphatic OH

The first step, step a), corresponds to a Fischer esterification between polyethylene glycol (PEG) (Mₙ=400 g·mol⁻¹, p=8-9, 1 eq, 5.00 g), 4,4-Bis(4-hydroxyphenyl)valeric acid (DPA) (2 eq, 7.16 g) in presence of p-toluene sulfonic acid (pTSA) introduced in catalytic amount (1 wt %). PEG, DPA, and pTSA were reacted together in melt at 130° C. and agitated by mechanical stirring for 24 hours, to provide 4,4-Bis(4-hydroxyphenyl)valeric ester terminated polyethylene glycol (PEG-DPA).

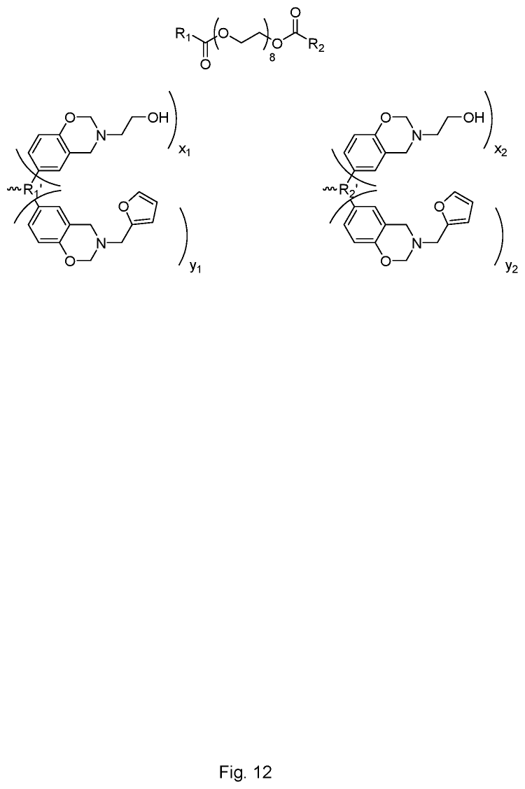

The second step, step b), corresponds to a Mannich condensation between 4,4-Bis(4-hydroxyphenyl)valeric ester terminated polyethylene glycol (PEG-DPA) (1 eq, 5.00 g), furfurylamine (fa) (1.0-2.0-3.0 eq, 0.52-1.04-1.56 g), mono-ethanolamine (mea) (3.0-2.0-1.0 eq, 0.98-0.65-0.33 g) and paraformaldehyde (PFA) (8 eq, 1.28 g). All these reactants were reacted together in melt at 70° C. and agitated by mechanical stirring for 24 hours to provide ester-containing benzoxazine monomers named respectively PEG-DPA-mea75/fa25 (1.0 eq fa, 3.0 eq. mea), PEG-DPA-mea50/fa50 (2.0 eq fa, 2.0 eq. mea), and PEG-DPA-mea25/fa75 (3.0 eq fa, 1.0 eq. mea) (FIG. 12).

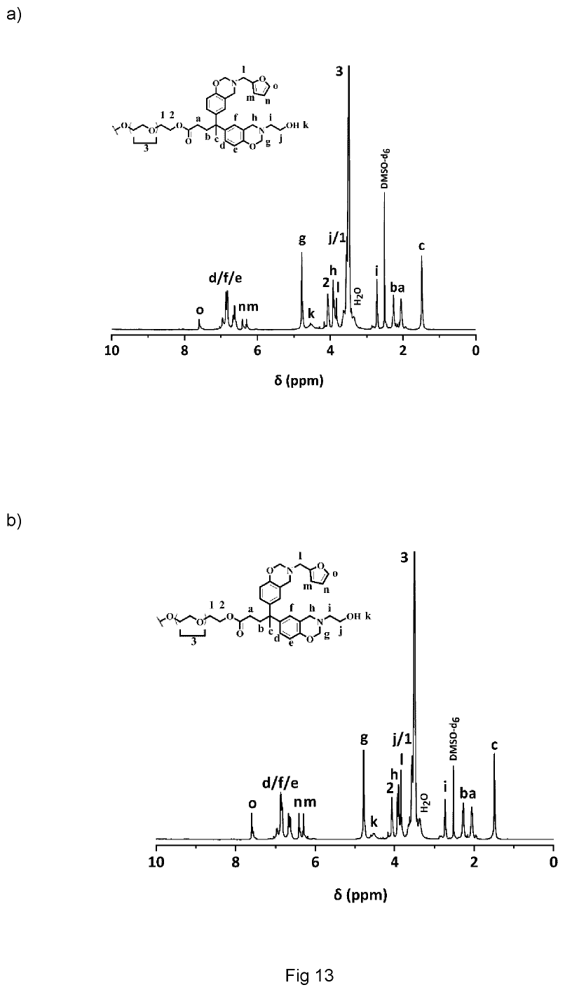

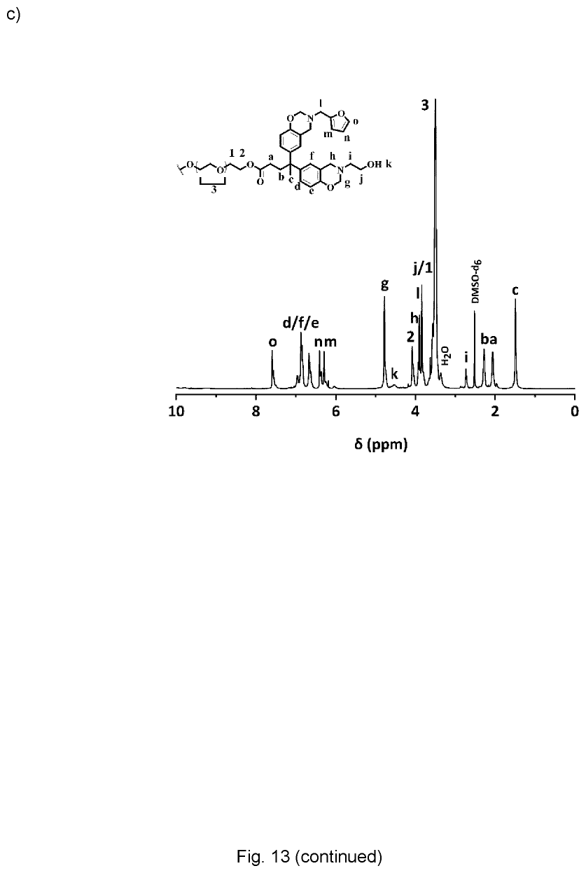

The FIG. 13 is displaying the ¹H NMR spectrum (AVANCE III HD Bruker spectrometer) of NMR spectrum of a) PEG-DPA-mea75/fa25, b) PEG-DPA-mea50/fa50, and c) PEG-DPA-mea25/fa75 ester-containing benzoxazine monomers.

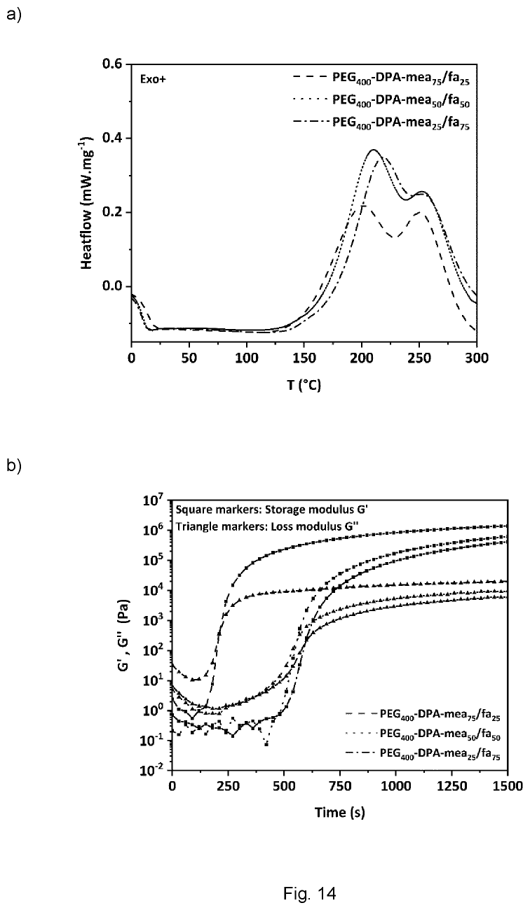

DSC curves in FIG. 14.a show an exothermic peak starting at a temperature of 123, 127 and 135° C. for PEG-DPA-mea75/fa25, PEG-DPA-mea50/fa50, and PEG-DPA-mea25/fa75, respectively. This peak corresponds to the ring opening of the benzoxazine rings upon heating. The second peak corresponds to the thermal decomposition of the ester linkage.

The curing of the PEG-DPA-mea/fa ester-containing benzoxazine monomers was monitored by rheological measurement in FIG. 14.b. The rheogram is performed under the following conditions: 1 Hz, with linear amplitude from 1 to 0.1%; 25 mm plates. The test is performed following a heating ramp from 80° C. to 140° C. at 15° C.·min⁻¹ followed by an isothermal measurement at 140° C. The storage and loss modulus are recorded as a function of time. The term “gelation time” is defined as the time when the storage and the loss modulus of the soften monomer increases abruptly to transform into a gel. The gelation is defined by the crossover point between the storage and the loss modulus. At 140° C., the gelation time is reached after 2012, 3172 and 3410 s, respectively for PEG-DPA-mea75/fa25, PEG-DPA-mea50/fa50, and PEG-DPA-mea25/fa75.

### Example 12: Vitrimer Synthesis from PEG-DPA-mea/fa Benzoxazine Monomers

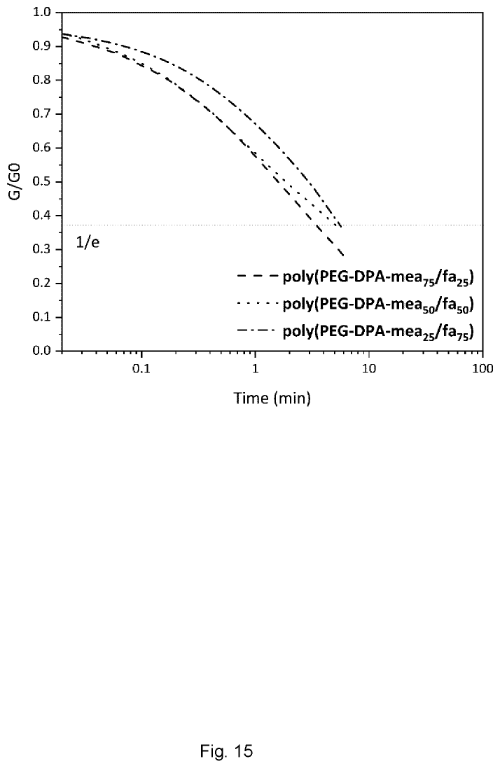

The PEG-DPA-mea/fa benzoxazine monomer from Example 11 was cured 1 h at 150° C. and 0.5 h at 170° C., allowing the benzoxazine rings to open and to react on themselves to form a 3D network vitrimer in a disk shape. The vitrimer behaviour of this sample was demonstrated through several rheology experiment. Viscoelastic properties of PEG-DPA-mea/fa vitrimers were studied by stress relaxation experiments recorded on Anton Paar Physica MCR 302 rheometer in plate-plate mode at 1% shear strain (FIG. 15). The relaxation time of the polymer was clearly noticeable and was recorded at 33.5, 52.9, and 56.8 min at 150° C. for poly(PEG-DPA-mea75/fa25), poly(PEG-DPA-mea50/fa50), and poly(PEG-DPA-mea25/fa75), respectively.

1.-14. (canceled)

15. An ester containing benzoxazine monomer of formula (I) 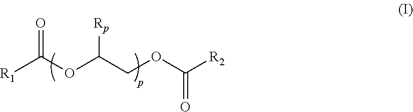
wherein
R₁ is 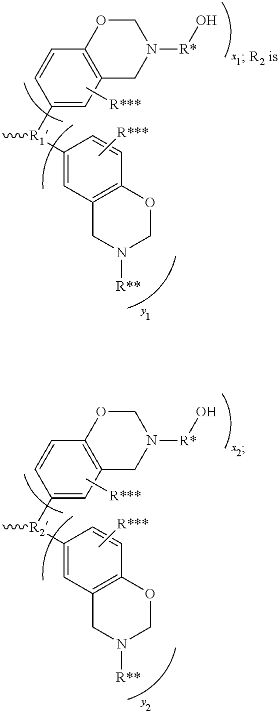
and
Rₚ is selected from the group consisting of H, a linear or branched C₁-C₆ alkyl or alkoxy group, a linear or branched C₂-C₆ alkenyl or alkylenoxy group, a substituted or unsubstituted linear or branched C₂-C₆ alkynyl group, a linear or branched C₁-C₆ alkyl or C₂-C₆ alkenyl substituted or unsubstituted phenyl group and 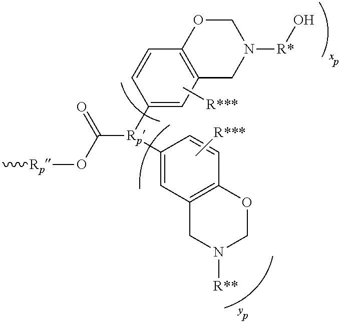
R₁ and R₂ of formula (I) are different;
x₁, x₂ and xₚ, independently, are of from 0 to 1 and are not together 0;
y₁=1-x₁; y₂=1-x₂ and yₚ=1-xₚ;
p is 1-100;
wherein
$${x}_{1} = \frac{{n}_{aminoalcohol ( R 1 )}}{{n}_{amines ( R 1 )}^{total}} \\ {x}_{2} = \frac{{n}_{aminoalcohol ( R 2 )}}{{n}_{amines ( R 2 )}^{total}} \\ {x}_{p} = \frac{{n}_{aminoalcohol ( Rp )}}{{n}_{amines ( Rp )}^{total}} \\ {y}_{1} = \frac{{n}_{amines ( R 1 )}}{{n}_{amines ( R 1 )}^{total}} \\ {y}_{2} = \frac{{n}_{amines ( R 2 )}}{{n}_{amines ( R 2 )}^{total}} \\ {y}_{p} = \frac{{n}_{amines ( Rp )}}{{n}_{amines ( Rp )}^{total}}$$
wherein n_{amine(R1)}^{total}=n_{amines(R1)}+n_{aminoalcohol(R1)}, and n_{aminoalcohol(R1)} being the number of aminoalcohol per R₁ group, n_{amines(R1)} represent the number of amines (excepting the number of aminoalcohol) per group R₁ and n_{amine(R1)}^{total}=n_{amines(R1)}+n_{aminoalcohol(R1)} is the total number of amino groups per group R₁;
wherein n_{amine(R2)}^{total}=n_{amines(R2)}+n_{aminoalcohol(R2)}, and n_{aminoalcohol(R1)} being the number of aminoalcohol per R₂ group, n_{amines(R2)} represents the number of amines (excepting the number of aminoalcohol) per group R₂ and n_{amine(R2)}^{total}=n_{amines(R2)}+n_{aminoalcohol(R2)} is the total number of amino groups per group R₂;
wherein n_{amine(Rp)}^{total}=n_{amines(Rp)}+n_{aminoalcohol(Rp)}, and n_{aminoalcohol(Rp)} being the number of aminoalcohol per Rₚ group, n_{amines(Rp)} represents the number of amines (excepting the number of aminoalcohol) per group Rₚ and n_{amine(Rp)}^{total}=n_{amines(Rp)}+n_{aminoalcohol(Rp)} is the total number of amino groups per group Rₚ,
R₁′, R₂′, and Rₚ′, independently, are selected from the group consisting of a —C-linear or branched C₁-C₆ alkyl or alkoxy group, a —C-linear or branched C₂-C₆ alkenyl or alkylenoxy group, a —C-substituted or unsubstituted linear or branched C₂-C₆ alkynyl group, and a —C-linear or branched C₁-C₆ alkyl or C₂-C₆ alkenyl substituted or unsubstituted phenyl group;
Rₚ″ is selected from the group consisting of a linear or branched C₁-C₆ alkyl or alkoxy group, a linear or branched C₂-C₆ alkenyl or alkylenoxy group, a substituted or unsubstituted linear or branched C₂-C₆ alkynyl group and a linear or branched C₁-C₆ alkyl or C₂-C₆ alkenyl substituted or unsubstituted phenyl group;
R* is selected from the group consisting of a linear or branched C₁-C₆ alkyl or alkoxy group, a cyclo(C₃-C₆alkyl) group, a heteocyclo(C₃-C₆alkyl) group, wherein the hetero atom is selected from N, S, and O, a linear or branched C₂-C₆ alkenyl or alkylenoxy group, a substituted or unsubstituted linear or branched C₂-C₆ alkynyl group, a linear or branched C₁-C₆ alkyl or C₂-C₆ alkenyl substituted or unsubstituted phenyl group, a (CH₂)ₙ₃-phenyl group and a —(CH₂)ₙ₃—O—(CH₂)ₙ₄ group, wherein n3 and n4, independently, are an integer from 1 to 10;
R** is the same as R* and further includes a member selected from the group consisting of a O—, N— or S—(CH₂)ₙ₃—CH—(CH₃)₂ group, a O—, N— or S—(CH₂)ₙ₃—(CHZ)ₙ₄—(CH₃)₂ group, a O—, N— or S—(CH₂)ₙ₃—(CHZ)ₙ₄—(CH₂)ₙ₃—CH₃ group, a O—, N— or S—(CHZ)ₙ₄—(CH₂)ₙ₃—CH₃ group, a O—, N— or S—(CHZ)ₙ₄—[(CH₂)ₙ₃—CH₃]₂ group, a O-substituted or unsubstituted C₂-C₆ linear or branched alkynyl group, a —(CH₂)ₙ₃—C≡N group and a polycyclic aromatic or a heteroaromatic hydrocarbon, such as naphthalene, anthracene, fluorene, phenanthrene, optionally substituted by a linear or branched C₁-C₆ alkyl or alkoxy group, a cyclo(C₃-C₆alkyl) group, a heterocyclo(C₃-C₆alkyl) group, a linear or branched C₂-C₆ alkenyl or alkylenoxy group, or by a substituted or unsubstituted linear or branched C₂-C₆ alkynyl group, wherein n3 and n4, independently, are an integer from 1 to 10, Z being selected from the group consisting of a linear or branched C₁-C₆ alkyl or alkoxy group, a linear or branched C₂-C₆ alkenyl or alkylenoxy group and a linear or branched C₁-C₆ alkyl or C₂-C₆ alkenyl substituted or unsubstituted phenyl group, and at least one O atom is present or not between two adjacent C,
R*** is selected from the group consisting of H, OH and a O-linear or branched C₁-C₆ alkyl group, and further includes a linear or branched C₁-C₁₅ alkyl group or a C₂-C₁₅ alkenyl group or

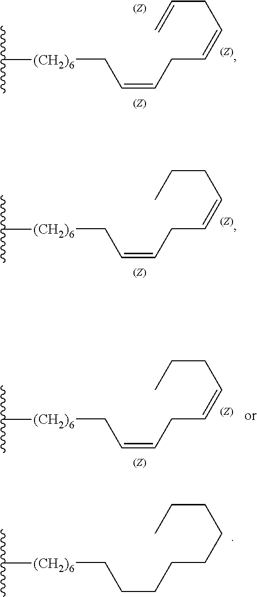

16. The ester containing benzoxazine monomer according to claim 15, wherein
R* is selected from the group consisting of a linear or branched C₁-C₄ alkyl or alkoxy group, a linear or branched C₂-C₄ alkenyl or alkylenoxy group, an unsubstituted linear or branched C₂-C₄ alkynyl group, an unsubstituted phenyl group and a (CH₂)ₙ₃-phenyl group, a —(CH₂)ₙ₃—O—(CH₂)ₙ₄ group, wherein n3 and n4, independently, are an integer from 1 to 6;
R** is the same as R* and may further include a member selected from a O—, N— or S—(CH₂)ₙ₃—CH—(CH₃)₂ group, a O—, N— or S—(CH₂)ₙ₃—(CHZ)ₙ₄—(CH₃)₂ group, a O—, N— or S—(CH₂)ₙ₃—(CHZ)ₙ₄—(CH₂)ₙ₃—CH₃ group, a O—, N— or S—(CHZ)ₙ₄—(CH₂)ₙ₃—CH₃ group, a O—, N— or S—(CHZ)ₙ₄—[(CH₂)ₙ₃—CH₃]₂ group, a O-substituted or unsubstituted C₂-C₄ linear or branched alkynyl group, a —(CH₂)ₙ₃—C≡N group and a polycyclic aromatic or a heteroaromatic hydrocarbon, wherein the hetero atom is selected from N, S, and O, such as naphthalene, anthracene, fluorene, furane, which is optionally substituted by a linear or branched C₁-C₄ alkyl or alkoxy group, a linear or branched C₂-C₄ alkenyl or alkylenoxy group, a cyclo(C₃-C₄alkyl) group, a heteocyclo(C₃-C₄ alkyl) group, or by a substituted or unsubstituted linear or branched C₂-C₄ alkynyl group, wherein n3 and n4, independently, are an integer from 1 to 6, Z being as defined in claim 1;
R*** is selected from the group consisting of H, OH and a O-linear or branched C₁-C₄ alkyl group, and further including a linear or branched C₁-C₁ alkyl group or C₂-C₁₀ alkenyl group or

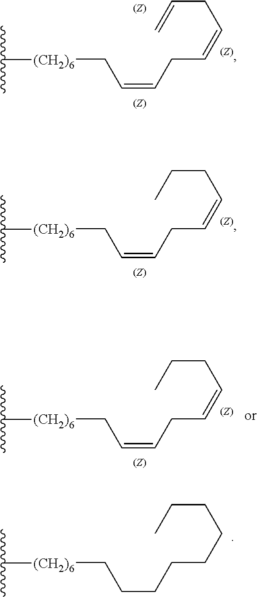

17. The ester containing benzoxazine monomer according to claim 15, wherein
R* is selected from the group consisting of groups —CH₃, —(CH₂)ₙ₃—CH₃, —(CH₂)ₙ₃—CH—[(CH₂)ₙ₄—CH₃]₂, —C(CH₃)₃, (CH₂)ₙ₃—(C₆H₅), —(CH₂)ₙ₃—CH═CH₂, —(CH₂)ₙ₃—C≡CH, —(CH₂)ₙ₃—O—(CH₂)ₙ₄ wherein n3 and n4 independently are integer from 1 to 4, phenyl, and —(CH₂)₃-phenyl;
R** is the group R*, or is selected from the group consisting of groups CH₃, —(CH₂)ₙ₃—CH₃, —(CH₂)ₙ₃—CH—[(CH₂)ₙ₄—CH₃]₂, —C(CH₃)₃, (CH₂)ₙ₃—(C₆H₅), —(CH₂)ₙ₃—CH═CH₂, —(CH₂)ₙ₃—C≡CH, O—(CH₂)ₙ₃—C≡CH, O—(CH₂)ₙ₃—C≡N, (CH₂)ₙ₃—C≡N, and —(CH₂)ₙ₃-substituted or unsubstituted furan, phenyl, and wherein n3 and n4, independently, are integer from 1 to 4;
R*** is selected from the group consisting of H, OH and a O-linear or branched C₁-C₃ alkyl group, and further includes a linear or branched C₁-C₆ alkyl group or C₂-C₆ alkenyl group or

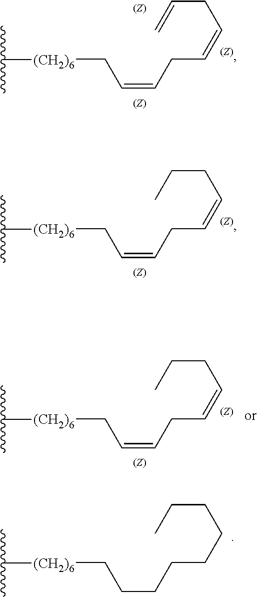

18. A process for synthesizing an ester-containing benzoxazine monomer of formula (I) 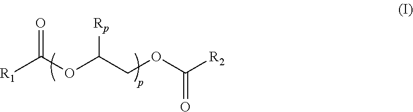
comprising the following steps consisting of:
a) reacting a phenolic acid derivative of formula (II), comprising at least one R*** group on the phenolic ring: 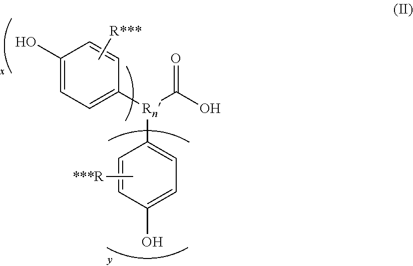
wherein x is of from 0 to 1, and y=1-x,
with a polyfunctional molecule or oligomer of formula (III) 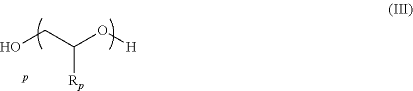
at a temperature of from 25° C. to 200° C., during 1 h-72 h, in the presence of a catalyst of Bronsted acid type, resulting in a phenol terminated oligomer or molecule (IV), and
b) reacting the compound (IV) with a mixture of:
an amino-alcohol of formula (V): 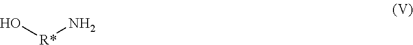
a primary amine derivative of formula (VI),
R**—NH₂ (VI), and
paraformaldehyde of formula (VII) 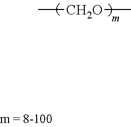
at a temperature range of from 80° C. to 100° C., from 1 h to 10 h, under stirring, for obtaining the compound of formula (I);
wherein R₁′, R₂′, Rₚ, R*, R**, R***, x₁, x₂, xₚ, y₁, y₂, yₚ, and p are, independently, as defined in claim 16, Rₙ′ being R₁′ or R₂′, R₁′ being different of R₂′, with the proviso that when at least one R*** of the phenolic acid derivative is in ortho position with regard to —OH group, then R*** is H.

19. The process according to claim 18, wherein the phenolic acid derivative (formula (II)) is selected from the group consisting of mono-, di-, tri-hydroxybenzoic acid derivatives, anacardic acid derivatives, hydroxycinnamic acid derivatives, aliphatic X-hydroxyphenyl acid derivatives, wherein X is 2-4 and aliphatic diphenolic acid derivatives, or mixtures thereof.

20. The process according to claim 18, wherein the respective stoichiometry of starting reactants on step a), phenolic acid derivative:polyfonctional molecule or oligomer is 1.0-3.0 eq.:1.0 eq, resulting in an 1.0 eq. of phenol terminated oligomer or molecule.

21. The process according to claim 18, wherein the primary amine derivatives are selected from the group consisting in allylamine, methylamine, ethylamine, propylamine, butylamine, isopropylamine, hexylamine, cyclohexylamine, stearylamine, 2-aminofluorene, aminophenyl acetylene, propargyl ether aniline, 4-aminobenzonitrile, furfurylamine and aniline, or mixtures thereof.

22. The process according to claim 18, wherein the temperature range of step b) is of from 80° C. to 95° C.

23. The process according to claim 18, wherein the step b) is performed from 1 h to 8 h, for the highest yield of at least 75%.

24. The process according to claim 18, wherein the respective stoichiometry of starting reactants on step b), phenol terminated oligomer or molecule:amino-alcohol:primary amine derivative:paraformaldehyde is 1.0 eq.:x₁ (1.0 eq-18.0 eq):y₁ (1.0 eq-18.0 eq):2.0-36.0 eq; or 1.0 eq.:x₂ (1.0 eq-18.0 eq): y₂ (1.0 eq-18.0 eq):2.0-36.0 eq; or 1.0 eq.:xₚ (1.0 eq-18.0 eq): yₚ (1.0 eq-18.0 eq):2.0-36.0 eq resulting in an 1.0 eq. of the ester-containing benzoxazine monomer, wherein x₁, x₂ and xₚ, independently, =0-1, and y₁=1-x₁, y₂=1-x₂ and yₚ=1-xₚ.

25. The process according to claim 18, wherein the relative molar % of amino-alcohol vs the relative molar % of primary amine derivative is 10 molar % vs 90 molar % respectively.

26. A process for preparing a polybenzoxazine derivative vitrimer comprising the step of polymerization of an ester-containing benzoxazine monomer of formula (I) 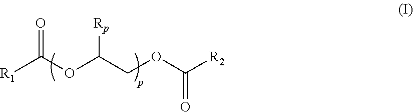
wherein
R₁ is 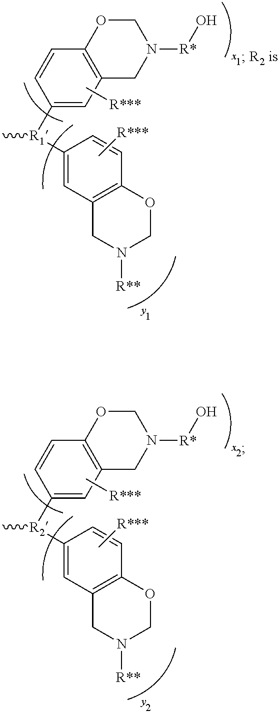
and
Rₚ is selected from the group consisting of H, a linear or branched C₁-C₆ alkyl or alkoxy group, a linear or branched C₂-C₆ alkenyl or alkylenoxy group, a substituted or unsubstituted linear or branched C₂-C₆ alkynyl group, a linear or branched C₁-C₆ alkyl or C₂-C₆ alkenyl substituted or unsubstituted phenyl group and 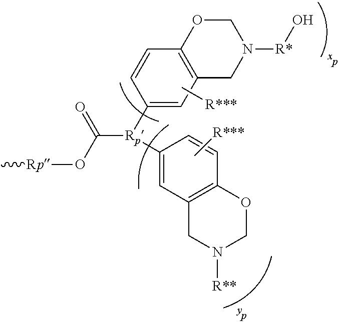
R₁ and R₂ of formula (I) are different;
x₁, x₂ and xₚ, independently, are of from 0 to 1 and are not together 0;
y₁=1-x₁; y₂=1-x₂ and yₚ=1-xₚ;
p is 1-100;
wherein
$${x}_{1} = \frac{{n}_{aminoalcohol ( R 1 )}}{{n}_{amines ( R 1 )}^{total}} \\ {x}_{2} = \frac{{n}_{aminoalcohol ( R 2 )}}{{n}_{amines ( R 2 )}^{total}} \\ {x}_{p} = \frac{{n}_{aminoalcohol ( Rp )}}{{n}_{amines ( Rp )}^{total}} \\ {y}_{1} = \frac{{n}_{amines ( R 1 )}}{{n}_{amines ( R 1 )}^{total}} \\ {y}_{2} = \frac{{n}_{amines ( R 2 )}}{{n}_{amines ( R 2 )}^{total}} \\ {y}_{p} = \frac{{n}_{amines ( Rp )}}{{n}_{amines ( Rp )}^{total}}$$
wherein n_{amine(R1)}^{total}=n_{amines(R1)}+n_{aminoalcohol(R1)}, and n_{aminoalcohol(R1)} being the number of aminoalcohol per R₁ group, n_{amines(R1)} represent the number of amines (excepting the number of aminoalcohol) per group R₁ and n_{amine(R1)}^{total}=n_{amines(R1)}+n_{aminoalcohol(R1)} is the total number of amino groups per group R₁;
wherein n_{amine(R2)}^{total}=n_{amines(R2)}+n_{aminoalcohol(R2)}, and n_{aminoalcohol(R2)} being the number of aminoalcohol per R₂ group, n_{amines(R2)} represents the number of amines (excepting the number of aminoalcohol) per group R₂ and n_{amine(R2)}^{total}=n_{amines(R2)}+n_{aminoalcohol(R2)} is the total number of amino groups per group R₂;
wherein n_{amine(Rp)}^{total}=n_{amines(Rp)}+n_{aminoalcohol(Rp)}, and n_{aminoalcohol(Rp)} being the number of aminoalcohol per Rₚ group, n_{amines(Rp)} represents the number of amines (excepting the number of aminoalcohol) per group Rₚ and n_{amine(Rp)}^{total}=n_{amines(Rp)}+n_{aminoalcohol(Rp)} is the total number of amino groups per group Rₚ,
R₁′, R₂′, and Rₚ′, independently, are selected from the group consisting of a —C-linear or branched C₁-C₆ alkyl or alkoxy group, a —C-linear or branched C₂-C₆ alkenyl or alkylenoxy group, a —C-substituted or unsubstituted linear or branched C₂-C₆ alkynyl group, and a —C-linear or branched C₁-C₆ alkyl or C₂-C₆ alkenyl substituted or unsubstituted phenyl group;
Rₚ″ is selected from the group consisting of a linear or branched C₁-C₆ alkyl or alkoxy group, a linear or branched C₂-C₆ alkenyl or alkylenoxy group, a substituted or unsubstituted linear or branched C₂-C₆ alkynyl group and a linear or branched C₁-C₆ alkyl or C₂-C₆ alkenyl substituted or unsubstituted phenyl group;
R* is selected from the group consisting of a linear or branched C₁-C₆ alkyl or alkoxy group, a cyclo(C₃-C₆alkyl) group, a heteocyclo(C₃-C₆alkyl) group, wherein the hetero atom is selected from N, S, and O, a linear or branched C₂-C₆ alkenyl or alkylenoxy group, a substituted or unsubstituted linear or branched C₂-C₆ alkynyl group, a linear or branched C₁-C₆ alkyl or C₂-C₆ alkenyl substituted or unsubstituted phenyl group, a (CH₂)ₙ₃-phenyl group and a —(CH₂)ₙ₃—O—(CH₂)ₙ₄ group, wherein n3 and n4, independently, are an integer from 1 to 10;
R** is the same as R* and further includes a member selected from the group consisting of a O—, N— or S—(CH₂)ₙ₃—CH—(CH₃)₂ group, a O—, N— or S—(CH₂)ₙ₃—(CHZ)ₙ₄—(CH₃)₂ group, a O—, N— or S—(CH₂)ₙ₃—(CHZ)ₙ₄—(CH₂)ₙ₃—CH₃ group, a O—, N— or S—(CHZ)ₙ₄—(CH₂)ₙ₃—CH₃ group, a O—, N— or S—(CHZ)ₙ₄—[(CH₂)ₙ₃—CH₃]₂ group, a O-substituted or unsubstituted C₂-C₆ linear or branched alkynyl group, a —(CH₂)ₙ₃—C≡N group and a polycyclic aromatic or a heteroaromatic hydrocarbon, such as naphthalene, anthracene, fluorene, phenanthrene, optionally substituted by a linear or branched C₁-C₆ alkyl or alkoxy group, a cyclo(C₃-C₆alkyl) group, a heterocyclo(C₃-C₆alkyl) group, a linear or branched C₂-C₆ alkenyl or alkylenoxy group, or by a substituted or unsubstituted linear or branched C₂-C₆ alkynyl group, wherein n3 and n4, independently, are an integer from 1 to 10, Z being selected from the group consisting of a linear or branched C₁-C₆ alkyl or alkoxy group, a linear or branched C₂-C₆ alkenyl or alkylenoxy group and a linear or branched C₁-C₆ alkyl or C₂-C₆ alkenyl substituted or unsubstituted phenyl group, and at least one O atom is present or not between two adjacent C,
R*** is selected from the group consisting of H, OH and a O-linear or branched C₁-C₆ alkyl group, and further includes a linear or branched C₁-C₁₅ alkyl group or a C₂-C₁₅ alkenyl group or 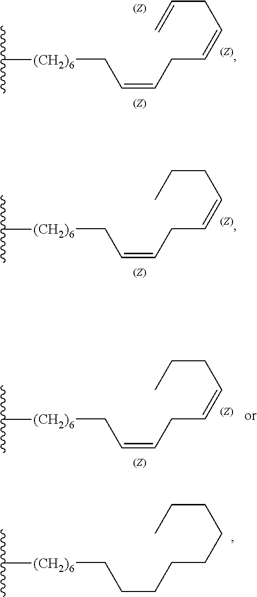
at temperatures within the range of from 100° C. to 250° C. for 1 h to 24 h, for obtaining polybenzoxazine derivatives vitrimers.
